(19) United States
(12) Patent Application Publication
(10) Pub. No.: US 2005/0020546 A1
(43) Pub. Date: Jan. 27, 2005

(54) Pharmaceutical compositions comprising active vitamin D compounds

(75) Inventors: Barbara F. Laidlaw, San Jose, CA (US); James Swarbrick, Pinehurst, NC (US)

Correspondence Address: STERNE, KESSLER, GOLDSTEIN & FOX PLLC, 1100 NEW YORK AVENUE, N.W., WASHINGTON, DC, 20005, US

(73) Assignee: Novacea, Inc.

(21) Appl. No.: 10/864,769

(22) Filed: Jun. 10, 2004

Related U.S. Application Data
(63) Continuation-in-part of application No. 10/841,954, filed on May 10, 2004.
(60) Provisional application No. 60/477,345, filed on Jun. 11, 2003.

(51) Int. Cl. A61K 31/59; A61K 31/355

(52) U.S. Cl. 514/167; 514/458

(57) ABSTRACT

Disclosed are pharmaceutical compositions comprising an active vitamin D compound in emulsion pre-concentrate formulations, as well as emulsions and sub-micron droplet emulsions produced therefrom. The compositions comprise a lipophilic phase component, one or more surfactants, and an active vitamin D compound. The compositions may optionally further comprise a hydrophilic phase component.

### BACKGROUND OF THE INVENTION

1. Field of the Invention

The present invention relates to novel pharmaceutical compositions comprising an active vitamin D compound, wherein the pharmaceutical compositions are emulsion pre-concentrates. The invention also relates to emulsions and sub-micron droplet emulsions produced upon dilution of the emulsion pre-concentrates with an aqueous solution.

2. Related Art

Vitamin D is a fat soluble vitamin which is essential as a positive regulator of calcium homeostasis. (See Harrison's Principles of Internal Medicine: Part Eleven, “Disorders of Bone and Mineral Metabolism,” Chapter 335, pp. 1860-1865, E. Braunwald et al., (eds.), McGraw-Hill, New York (1987)). The active form of vitamin D is 1α,25-dihydroxyvitamin D₃, also known as calcitriol. Specific nuclear receptors for active vitamin D compounds have been discovered in cells from diverse organs not involved in calcium homeostasis. (Miller et al., Cancer Res. 52:515-520 (1992)). In addition to influencing calcium homeostasis, active vitamin D compounds have been implicated in osteogenesis, modulation of immune response, modulation of the process of insulin secretion by the pancreatic B cell, muscle cell function, and the differentiation and growth of epidermal and hematopoietic tissues.

Moreover, there have been many reports demonstrating the utility of active vitamin D compounds in the treatment of cancer. For example, it has been shown that certain vitamin D compounds and analogues possess potent antileukemic activity by virtue of inducing the differentiation of malignant cells (specifically, leukemic cells) to non-malignant macrophages (monocytes) and are useful in the treatment of leukemia. (Suda et al., U.S. Pat. No. 4,391,802; Partridge et al., U.S. Pat. No. 4,594,340). Antiproliferative and differentiating actions of calcitriol and other vitamin D₃ analogues have also been reported with respect to the treatment of prostate cancer. (Bishop et al., U.S. Pat. No. 5,795,882). Active vitamin D compounds have also been implicated in the treatment of skin cancer (Chida et al., Cancer Research 45:5426-5430 (1985)), colon cancer (Disman et al., Cancer Research 47:21-25 (1987)), and lung cancer (Sato et al., Tohoku J. Exp. Med. 138:445-446 (1982)). Other reports suggesting important therapeutic uses of active vitamin D compounds are summarized in Rodriguez et al., U.S. Pat. No. 6,034,079.

Although the administration of active vitamin D compounds may result in substantial therapeutic benefits, the treatment of cancer and other diseases with such compounds is limited by the effects these compounds have on calcium metabolism. At the levels required in vivo for effective use as anti-proliferative agents, active vitamin D compounds can induce markedly elevated and potentially dangerous blood calcium levels by virtue of their inherent calcemic activity. That is, the clinical use of calcitriol and other active vitamin D compounds as anti-proliferative agents is precluded, or severely limited, by the risk of hypercalcemia.

It has been shown that the problem of systemic hypercalcemia can be overcome by “pulse-dose” administration of a sufficient dose of an active vitamin D compound such that an anti-proliferative effect is observed while avoiding the development of severe hypercalcemia. (U.S. Pat. No. 6,521,608). According to 6,521,608, the active vitamin D compound may be administered no more than every three days, for example, once a week at a dose of at least 0.12 μg/kg per day (8.4 μg in a 70 kg person). Pharmaceutical compositions used in the pulse-dose regimen of 6,521,608 comprise 5-100 μg of active vitamin D compound and may be administered in the form for oral, intravenous, intramuscular, topical, transdermal, sublingual, intranasal, intratumoral or other preparations.

ROCALTROL is the trade name of a calcitriol formulation sold by Roche Laboratories. ROCALTROL is available in the form of capsules containing 0.25 and 0.5 μg calcitriol and as an oral solution containing 1 μg/mL of calcitriol. All dosage forms contain butylated hydroxyanisole (BHA) and butylated hydroxytoluene (BHT) as antioxidants. The capsules also contain a fractionated triglyceride of coconut oil and the oral solution contains a fractionated triglyceride of palm seed oil. (Physician's Desk Reference, 54^{th} Edition, pp 2649-2651, Medical Economics Company, Inc., Montvale, N.J. (2000)).

It is known that calcitriol is light-sensitive and is especially prone to oxidation. Moreover, calcitriol and other active vitamin D compounds are lipophilic, meaning that they are soluble in lipids and some organic solvents, while being substantially insoluble or only sparsely soluble in water. Because of the lipophilic nature of active vitamin D compounds, the dispersion of such compounds in aqueous solutions, such as the gastric fluids of the stomach, is significantly limited. Accordingly, the pharmacokinetic parameters of active vitamin D compound formulations heretofore described in the art are sub-optimal for use with high dose pulse administration regimens. In addition, the active vitamin D compound formulations that are currently available tend to exhibit substantial variability of absorption in the small intestine. Moreover, for oral administration, the relationship between dosage and blood concentration that is observed with most active vitamin D compound formulations is not linear; that is, the quantity of compound absorbed into the blood stream does not correlate with the amount of compound that is administered in a given dose, especially at higher dosage levels.

Thus, there is a need for improved pharmaceutical compositions comprising active vitamin D compounds, particularly in the context of pulse-dose treatment regimens that are designed to provide anti-proliferative (e.g., anti-cancer) benefits while avoiding the consequence of hypercalcemia. In particular, a need exists in the art for a pharmaceutical composition comprising an active vitamin D compound that remains stable over prolonged periods of time, even at elevated temperatures, while at the same time exhibiting improved pharmacokinetic parameters for the active vitamin D compound, and reduced variability in absorption, when administered to a patient.

### BRIEF SUMMARY OF THE INVENTION

The present invention overcomes the disadvantages heretofore encountered in the art by providing pharmaceutical compositions comprising active vitamin D compounds in emulsion pre-concentrate formulations. The pharmaceutical compositions of the present invention are an advance over the prior art in that they provide a dosage form of active vitamin D compounds, such as calcitriol, in a sufficiently high concentration to permit convenient use, stability and rapid dispersion in solution, and yet meet the required criteria in terms of pharmacokinetic parameters, especially in the context of pulse-dosing administration regimens. More specifically, in a preferred embodiment, the pharmaceutical compositions of the present invention exhibit a Cₘₐₓ that is at least 1.5 to two times greater than the Cₘₐₓ that is observed with ROCALTROL, and a shorter Tₘₐₓ than that which is observed with ROCALTROL.

The emulsion pre-concentrates of the present invention are non-aqueous formulations for an active vitamin D compound that are capable of providing a pharmaceutically acceptable emulsion, upon contact with water or other aqueous solution.

According to one aspect of the invention, pharmaceutical compositions are provided comprising (a) a lipophilic phase component, (b) one or more surfactants, and (c) an active vitamin D compound; wherein said composition is an emulsion pre-concentrate, which upon dilution with water in a water to composition ratio of about 1:1 or more of water forms an emulsion having an absorbance of greater than 0.3 at 400 nm. According to this aspect of the invention, the pharmaceutical compositions may further comprise a hydrophilic phase component.

According to another aspect of the invention, a pharmaceutical emulsion composition is provided comprising water and an emulsion pre-concentrate, said emulsion pre-concentrate comprising (a) a lipophilic phase component, (b) one or more surfactants, and (c) an active vitamin D compound, and optionally, a hydrophobic phase component.

The emulsions produced from the emulsion pre-concentrates of the present invention (upon dilution with water) include both emulsions as conventionally understood by those of ordinary skill in the art (i.e., a dispersion of an organic phase in water), as well as “sub-micron droplet emulsions” (i.e., dispersions of an organic phase in water wherein the average diameter of the dispersion particles is less than 1000 nm.)

According to another aspect of the invention, methods are provided for the preparation of emulsion pre-concentrates comprising active vitamin D compounds. The methods encompassed within this aspect of the invention comprise bringing an active vitamin D compound, e.g., calcitriol, into intimate admixture with a lipophilic phase component and with one or more surfactants, and optionally, with a hydrophilic phase component.

In yet another aspect of the invention, methods are provided for the treatment and prevention of hyperproliferative diseases such as cancer and psoriasis, said methods comprising administering an active vitamin D compound in an emulsion pre-concentrate formulation to a patient in need thereof. Alternatively, the active vitamin D compound can be administered in an emulsion formulation that is made by diluting an emulsion pre-concentrate of the present invention with an appropriate quantity of water. In a preferred embodiment of this aspect of the invention, the administration of the active vitamin D compound to a patient is accomplished by using, e.g., a pulse dosing regimen. For example, according to this aspect of the invention, an active vitamin D compound in an emulsion pre-concentrate formulation is administered to a patient no more than once every three days at a dose of at least 0.12 μg/kg per day.

### BRIEF DESCRIPTIONS OF THE FIGURES

FIG. 1 is a graphical representation of the mean plasma concentration of calcitriol in dogs versus time following administration of three different formulations of calcitriol at a dose of 1 μg/kg.

FIGS. 2A and 2B are graphical representations of the mean plasma concentration-time curve for calcitriol after escalating doses of semi-solid #3 in male (FIG. 2A) and female (FIG. 2B) dogs.

FIGS. 3A and 3B are graphical representations of the plasma concentration-time curve for calcitriol in male (FIG. 3A) and female (FIG. 3B) dogs after semi-solid #3 dosing.

FIGS. 4A and 4B are graphical representations of the mean serum calcium after increasing doses of semi-solid #3 in male (FIG. 4A) and female (FIG. 4B) dogs.

FIGS. 5A-5C are graphical representations of the plasma calcitriol and serum calcium data following administration of semi-solid #3 in male dogs.

FIG. 6 is a graphical representation of the mean plasma concentration of calcitriol by dose group in humans following administration of semi-solid #3.

### DETAILED DESCRIPTION OF THE INVENTION

The present invention is directed to pharmaceutical compositions comprising active vitamin D compounds in emulsion pre-concentrate formulations. The compositions of the invention meet or substantially reduce the difficulties associated with active vitamin D compound therapy hitherto encountered in the art including, in particular, undesirable pharmacokinetic parameters of the compound upon administration to a patient.

It has been found that the compositions of the invention permit the preparation of semi-solid and liquid compositions containing an active vitamin D compound in sufficiently high concentration to permit, e.g., convenient oral administration, while at the same time achieving improved pharmacokinetic parameters for the active vitamin D compound. For example, as compared to ROCALTROL, the compositions of the present invention exhibit a Cₘₐₓ that is at least 1.5 to two times greater than the Cₘₐₓ that is observed with ROCALTROL, and a shorter Tₘₐₓ than that which is observed with ROCALTROL. Preferably, the pharmaceutical compositions of the present invention provide a Cₘₐₓ of at least about 900 pg/mL plasma, more preferably about 900 to about 3000 pg/mL plasma, more preferably about 1500 to about 3000 pg/mL plasma. In addition, the compositions of the invention preferably provide a Tₘₐₓ of less than about 6.0 hours, more preferably about 1.0 to about 3.0 hours, more preferably about 1.5 to about 2.0 hours. In addition, the compositions of the invention preferably provide a T_{1/2} of less than about 25 hours, more preferably about 2 to about 10 hours, more preferably about 5 to about 9 hours.

The term Cₘₐₓ is defined as the maximum concentration of active vitamin D compound achieved in the serum following administration of the drug. The term Tₘₐₓ is defined as the time at which Cₘₐₓ is achieved. The term T_{1/2} is defined as the time required for the concentration of active vitamin D compound in the serum to decrease by half. The disclosed values for pharmacokinetic data apply to the population of recipients of a composition comprising an active vitamin D compound as a whole, not individual recipients. Thus, any individual receiving a composition of the present invention may not necessarily achieve the preferred pharmacokinetic parameters. However, when a composition of the present invention is administered to a sufficiently large population of subjects, the pharmacokinetic parameters will approximately match the values disclosed herein.

According to one aspect of the present invention, a pharmaceutical composition is provided comprising (a) a lipophilic phase component, (b) one or more surfactants, (c) an active vitamin D compound; wherein said composition is an emulsion pre-concentrate, which upon dilution with water, in a water to composition ratio of about 1:1 or more of said water, forms an emulsion having an absorbance of greater than 0.3 at 400 nm. The pharmaceutical composition of the invention may further comprise a hydrophilic phase component.

In another aspect of the invention, a pharmaceutical emulsion composition is provided comprising water (or other aqueous solution) and an emulsion pre-concentrate.

The term “emulsion pre-concentrate,” as used herein, is intended to mean a system capable of providing an emulsion upon contacting with, e.g., water. The term “emulsion,” as used herein, is intended to mean a colloidal dispersion comprising water and organic components including hydrophobic (lipophilic) organic components. The term “emulsion” is intended to encompass both conventional emulsions, as understood by those skilled in the art, as well as “sub-micron droplet emulsions,” as defined immediately below.

The term “sub-micron droplet emulsion,” as used herein is intended to mean a dispersion comprising water and organic components including hydrophobic (lipophilic) organic components, wherein the droplets or particles formed from the organic components have an average maximum dimension of less than about 1000 nm.

Sub-micron droplet emulsions are identifiable as possessing one or more of the following characteristics. They are formed spontaneously or substantially spontaneously when their components are brought into contact, that is without substantial energy supply, e.g., in the absence of heating or the use of high shear equipment or other substantial agitation.

The particles of a sub-micron droplet emulsion may be spherical, though other structures are feasible, e.g. liquid crystals with lamellar, hexagonal or isotropic symmetries. Generally, sub-micron droplet emulsions comprise droplets or particles having a maximum dimension (e.g., average diameter) of between about 50 nm to about 1000 nm, and preferably between about 200 nm to about 300 mm.

The term “pharmaceutical composition” as used herein is to be understood as defining compositions of which the individual components or ingredients are themselves pharmaceutically acceptable, e.g., where oral administration is foreseen, acceptable for oral use and, where topical administration is foreseen, topically acceptable.

The pharmaceutical compositions of the present invention will generally form an emulsion upon dilution with water. The emulsion will form according to the present invention upon the dilution of an emulsion pre-concentrate with water in a water to composition ratio of about 1:1 or more of said water. According to the present invention, the ratio of water to composition can be, e.g., between 1:1 and 5000:1. For example, the ratio of water to composition can be about 1:1, 2:1, 3:1, 4:1, 5:1, 10:1, 200:1, 300:1, 500:1, 1000:1, or 5000:1. The skilled artisan will be able to readily ascertain the particular ratio of water to composition that is appropriate for any given situation or circumstance.

According to the present invention, upon dilution of said emulsion pre-concentrate with water, an emulsion will form having an absorbance of greater than 0.3 at 400 nm. The absorbance at 400 nm of the emulsions formed upon 1:100 dilution of the emulsion pre-concentrates of the present invention can be, e.g., between 0.3 and 4.0. For example, the absorbance at 400 nm can be, e.g., about 0.4, 0.5, 0.6, 1.0, 1.2, 1.6, 2.0, 2.2, 2.4, 2.5, 3.0, or 4.0. Methods for determining the absorbance of a liquid solution are well known by those in the art. The skilled artisan will be able to ascertain and adjust the relative proportions of the ingredients of the emulsions pre-concentrates of the invention in order to obtain, upon dilution with water, an emulsion having any particular absorbance encompassed within the scope of the invention.

The pharmaceutical compositions of the present invention can be, e.g., in a semi-solid formulation or in a liquid formulation. Semi-solid formulations of the present invention can be any semi-solid formulation known by those of ordinary skill in the art, including, e.g., gels, pastes, creams and ointments.

The pharmaceutical compositions of the present invention comprise a lipophilic phase component. Suitable components for use as lipophilic phase components include any pharmaceutically acceptable solvent which is non-miscible with water. Such solvents will appropriately be devoid or substantially devoid of surfactant function.

The lipophilic phase component may comprise mono-, di- or triglycerides. Mono-, di- and triglycerides that may be used within the scope of the invention include those that are derived from C₆, C₈, C₁₀, C₁₂, C₁₄, C₁₆, C₁₈, C₂₀ and C₂₂ fatty acids. Exemplary diglycerides include, in particular, diolein, dipalmitolein, and mixed caprylin-caprin diglycerides. Preferred triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

A preferred triglyceride is the medium chain triglyceride available under the trade name LABRAFAC CC. Other preferred triglycerides include neutral oils, e.g., neutral plant oils, in particular fractionated coconut oils such as known and commercially available under the trade name MIGLYOL, including the products: MIGLYOL 810; MIGLYOL 812; MIGLYOL 818; and CAPTEX 355.

Also suitable are caprylic-capric acid triglycerides such as known and commercially available under the trade name MYRITOL, including the product MYRITOL 813. Further suitable products of this class are CAPMUL MCT, CAPTEX 200, CAPTEX 300, CAPTEX 800, NEOBEE M5 and MAZOL 1400.

Especially preferred as lipophilic phase component is the product MIGLYOL 812. (See U.S. Pat. No. 5,342,625).

Pharmaceutical compositions of the present invention may further comprise a hydrophilic phase component. The hydrophilic phase component may comprise, e.g., a pharmaceutically acceptable C₁₋₅ alkyl or tetrahydrofurfuryl di- or partial-ether of a low molecular weight mono- or poly-oxy-alkanediol. Suitable hydrophilic phase components include, e.g., di- or partial-, especially partial-, -ethers of mono- or poly-, especially mono- or di-, -oxy-alkanediols comprising from 2 to 12, especially 4 carbon atoms. Preferably the mono- or poly-oxy-alkanediol moiety is straight-chained. Exemplary hydrophilic phase components for use in relation to the present invention are those known and commercially available under the trade names TRANSCUTOL and COLYCOFUROL. (See U.S. Pat. No. 5,342,625).

In an especially preferred embodiment, the hydrophilic phase component comprises 1,2-propyleneglycol.

The hydrophilic phase component of the present invention may of course additionally include one or more additional ingredients. Preferably, however, any additional ingredients will comprise materials in which the active vitamin D compound is sufficiently soluble, such that the efficacy of the hydrophilic phase as an active vitamin D compound carrier medium is not materially impaired. Examples of possible additional hydrophilic phase components include lower (e.g., C₁₋₅) alkanols, in particular ethanol.

Pharmaceutical compositions of the present invention also comprise one or more surfactants. Surfactants that can be used in conjunction with the present invention include hydrophilic or lipophilic surfactants, or mixtures thereof. Especially preferred are non-ionic hydrophilic and non-ionic lipophilic surfactants.

Suitable hydrophilic surfactants include reaction products of natural or hydrogenated vegetable oils and ethylene glycol, i.e. polyoxyethylene glycolated natural or hydrogenated vegetable oils, for example polyoxyethylene glycolated natural or hydrogenated castor oils. Such products may be obtained in known manner, e.g., by reaction of a natural or hydrogenated castor oil or fractions thereof with ethylene oxide, e.g., in a molar ratio of from about 1:35 to about 1:60, with optional removal of free polyethyleneglycol components from the product, e.g., in accordance with the methods disclosed in German Auslegeschriften 1,182,388 and 1,518,819.

Suitable hydrophilic surfactants for use in the present pharmaceutical compounds also include polyoxyethylene-sorbitan-fatty acid esters, e.g., mono- and trilauryl, palmityl, stearyl and oleyl esters, e.g., of the type known and commercially available under the trade name TWEEN; including the products:
TWEEN 20 (polyoxyethylene(20)sorbitanmonolaurate),
TWEEN 40 (polyoxyethylene(20)sorbitanmonopalmitate),
TWEEN 60 (polyoxyethylene(20)sorbitanmonostearate),
TWEEN 80 (polyoxyethylene(20)sorbitanmonooleate),
TWEEN 65 (polyoxyethylene(20)sorbitantristearate),
TWEEN 85 (polyoxyethylene(20)sorbitantrioleate),
TWEEN 21 (polyoxyethylene(4)sorbitanmonolaurate),
TWEEN 61 (polyoxyethylene(4)sorbitanmonostearate), and
TWEEN 81 (polyoxyethylene(5)sorbitanmonooleate).

Especially preferred products of this class for use in the compositions of the invention are the above products TWEEN 40 and TWEEN 80. (See Hauer, et al., U.S. Pat. No. 5,342,625).

Also suitable as hydrophilic surfactants for use in the present pharmaceutical compounds are polyoxyethylene alkylethers; polyoxyethylene glycol fatty acid esters, for example polyoxyethylene stearic acid esters; polyglycerol fatty acid esters; polyoxyethylene glycerides; polyoxyethylene vegetable oils; polyoxyethylene hydrogenated vegetable oils; reaction mixtures of polyols and, e.g., fatty acids, glycerides, vegetable oils, hydrogenated vegetable oils, and sterols; polyoxyethylene-polyoxypropylene co-polymers; polyoxyethylene-polyoxypropylene block co-polymers; dioctylsuccinate, dioctylsodiumsulfosuccinate, di-[2-ethylhexyl]-succinate or sodium lauryl sulfate; phospholipids, in particular lecithins such as, e.g., soya bean lecithins; propylene glycol mono- and di-fatty acid esters such as, e.g., propylene glycol dicaprylate, propylene glycol dilaurate, propylene glycol hydroxystearate, propylene glycol isostearate, propylene glycol laurate, propylene glycol ricinoleate, propylene glycol stearate, and, especially preferred, propylene glycol caprylic-capric acid diester; and bile salts, e.g., alkali metal salts, for example sodium taurocholate.

Suitable lipophilic surfactants include alcohols; polyoxyethylene alkylethers; fatty acids; bile acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid esters of mono/diglycerides; propylene glycol diglycerides; sorbitan fatty acid esters; polyoxyethylene sorbitan fatty acid esters; polyoxyethylene-polyoxypropylene block copolymers; trans-esterified vegetable oils; sterols; sugar esters; sugar ethers; sucroglycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and mixtures thereof.

Suitable lipophilic surfactants for use in the present pharmaceutical compounds also include trans-esterification products of natural vegetable oil triglycerides and polyalkylene polyols. Such trans-esterification products are known in the art and may be obtained e.g., in accordance with the general procedures described in U.S. Pat. No. 3,288,824. They include trans-esterification products of various natural (e.g., non-hydrogenated) vegetable oils for example, maize oil, kernel oil, almond oil, ground nut oil, olive oil and palm oil and mixtures thereof with polyethylene glycols, in particular polyethylene glycols having an average molecular weight of from 200 to 800. Preferred are products obtained by trans-esterification of 2 molar parts of a natural vegetable oil triglyceride with one molar part of polyethylene glycol (e.g., having an average molecular weight of from 200 to 800). Various forms of trans-esterification products of the defined class are known and commercially available under the trade name LABRAFIL.

Additional lipophilic surfactants that are suitable for use with the present pharmaceutical compositions include oil-soluble vitamin derivatives, e.g., tocopherol PEG-1000 succinate (“vitamin E TPGS”).

Also suitable as lipophilic surfactants for use in the present pharmaceutical compounds are mono-, di- and mono/di-glycerides, especially esterification products of caprylic or capric acid with glycerol; sorbitan fatty acid esters; pentaerythritol fatty acid esters and polyalkylene glycol ethers, for example pentaerythrite- -dioleate, -distearate, -monolaurate, -polyglycol ether and -monostearate as well as pentaerythrite-fatty acid esters; monoglycerides, e.g., glycerol monooleate, glycerol monopalmitate and glycerol monostearate; glycerol triacetate or (1,2,3)-triacetin; and sterols and derivatives thereof, for example cholesterols and derivatives thereof, in particular phytosterols, e.g., products comprising sitosterol, campesterol or stigmasterol, and ethylene oxide adducts thereof, for example soya sterols and derivatives thereof.

It is understood by those of ordinary skill in the art that several commercial surfactant compositions contain small to moderate amounts of triglycerides, typically as a result of incomplete reaction of a triglyceride starting material in, for example, a trans-esterification reaction. Thus, the surfactants that are suitable for use in the present pharmaceutical compositions include those surfactants that contain a triglyceride. Examples of commercial surfactant compositions containing triglycerides include some members of the surfactant families GELUCIRES, MASINES, AND IMWITORS. Specific examples of these compounds are GELUCIRE 44/14 (saturated polyglycolized glycerides); GELUCIRE 50/13 (saturated polyglycolized glycerides); GELUCIRE 53/10 (saturated polyglycolized glycerides); GELUCIRE 33/01 (semi-synthetic triglycerides of C₈-C₁₈ saturated fatty acids); GELUCIRE 39/01 (semi-synthetic glycerides); other GELUCIRE, such as 37/06, 43/01, 35/10, 37/02, 46/07, 48/09, 50/02, 62/05, etc.; MASINE 35-I (linoleic glycerides); and IMWITOR 742 (caprylic/capric glycerides). (See U.S. Pat. No. 6,267,985).

Still other commercial surfactant compositions having significant triglyceride content are known to those skilled in the art. It should be appreciated that such compositions, which contain triglycerides as well as surfactants, may be suitable to provide all or part of the lipophilic phase component of the of the present invention, as well as all or part of the surfactants.

The pharmaceutical compositions of the present invention also comprise an active vitamin D compound. The term “active vitamin D compound,” as used herein, is intended to refer to vitamin D which has been hydroxylated in at least the carbon-1 position of the A ring, e.g., 1α-hydroxyvitamin D3. The preferred active vitamin D compound in relation to the composition of the present invention is 1α,25-hydroxyvitamin D₃, also known as calcitriol. A large number of other active vitamin D compounds are known and can be used in the practice of the invention. Examples include 1α-hydroxy derivatives with a 17 side chain greater in length than the cholesterol or ergosterol side chains (see U.S. Pat. No. 4,717,721); cyclopentano-vitamin D analogs (see U.S. Pat. No. 4,851,401); vitamin D₃ analogues with alkynyl, alkenyl, and alkanyl side chains (see U.S. Pat. Nos. 4,866,048 and 5,145,846); trihydroxycalciferol (see U.S. Pat. No. 5,120,722); fluoro-cholecalciferol compounds (see U.S. Pat. No. 5,547,947); methyl substituted vitamin D (see U.S. Pat. No. 5,446,035); 23-oxa-derivatives (see U.S. Pat. No. 5,411,949); 19-nor-vitamin D compounds (see U.S. Pat. No. 5,237,110); and hydroxylated 24-homo-vitamin D derivatives (see U.S. Pat. No. 4,857,518). Particular examples include ROCALTROL (Roche Laboratories); CALCIJEX injectable calcitriol; investigational drugs from Leo Pharmaceuticals including EB 1089 (24a,26a,27a-trihomo-22,24-diene-1αa,25-(OH)₂-D₃, KH 1060 (20-epi-22-oxa-24a,26a,27a-trihomo-1α,25-(OH)₂-D₃), Seocalcitol, MC 1288 (1,25-(OH)₂-20-epi-D₃) and MC 903 (calcipotriol, 1α,24s-(OH)₂-22-ene-26,27-dehydro-D₃); Roche Pharmaceutical drugs that include 1,25-(OH)₂-16-ene-D₃, 1,25-(OH)₂-16-ene-23-yne-D₃, and 25-(OH)₂-16-ene-23-yne-D₃; Chugai Pharmaceuticals 22-oxacalcitriol (22-oxa-1α,25-(OH)₂-D₃; 1α-(OH)-D₅ from the University of Illinois; and drugs from the Institute of Medical Chemistry-Schering AG that include ZK 161422 (20-methyl-1,25-(OH)₂-D₃) and ZK 157202 (20-methyl-23-ene-1,25-(OH)₂-D₃); 1α-(OH)-D₂; 1α-(OH)-D₃ and 1α-(OH)-D₄. Additional examples include 1α,25-(OH)₂-26,27-d₆-D₃; 1α,25-(OH)₂-22-ene-D₃; 1α,25-(OH)₂-D₃; 1α,25-(OH)₂-D₂; 1α,25-(OH)₂-D₄; 1α,24,25-(OH)₃-D₃; 1α,24,25-(OH)₃-D₂; 1α,24,25-(OH)₃-D₄; 1α-(OH)-25-FD₃; 1α-(OH)-25-FD₄; 1α-(OH)-25-FD₂; 1α,24-(OH)₂-D₄; 1α,24-(OH)₂-D₃; 1α,24-(OH)₂-D₂; 1α,24-(OH)₂-25-FD₄; 1α,24-(OH)₂-25-FD₃; 1α,24-(OH)₂-25-FD₂; 1α,25-(OH)₂-26,27-F₆-22-ene-D₃; 1α,25-(OH)₂-26,27-F₆-D₃; 1α,25S—(OH)₂-26-F₃-D₃; 1α,25-(OH)₂-24-F₂-D₃; 1α,25S,26-(OH)₂-22-ene-D₃; 1α,25R,26-(OH)₂-22-ene-D₃; 1α,25-(OH)₂-D₂; 1α,25-(OH)₂-24-epi-D₃; 1α,25-(OH)₂-23-yne-D₃; 1α,25-(OH)₂-24R—F-D₃; 1α,25S,26-(OH)₂-D₃; 1,24R—(OH)₂-25F-D₃; 1α,25-(OH)₂-26,27-F₆-23-yne-D₃; 1α,25R—(OH)₂-26-F₃-D₃; 1α,25,28-(OH)₃-D₂; 1α,25-(OH)₂-16-ene-23-yne-D₃; 1α,24R,25-(OH)₃-D₃; 1,25-(OH)₂-26,27-F₆-23-ene-D₃; 1α,25R—(OH)₂-22-ene-26-F₃-D₃; 1α,25S—(OH)₂-22-ene-26-F₃-D₃; 1α,25R—(OH)₂-D₃-26,26,26-d₃; 1α,25S—(OH)₂-D₃-26,26,26-d₃; and 1α,25R—(OH)₂-22-ene-D₃-26,26,26-d₃. Additional examples can be found in U.S. Pat. No. 6,521,608. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

In a preferred embodiment of the invention, the active vitamin D compound has a reduced hypercalcemic effect as compared to vitamin D so that increased doses of the compound can be administered without inducing hypercalcemia in the animal. A reduced hypercalcemic effect is defined as an effect which is less than the hypercalcemic effect induced by administration of an equal dose of 1α,25-hydroxyvitamin D₃ (calcitriol). As an example, EB 1089 has a hypercalcemic effect which is 50% of the hypercalcemic effect of calcitriol. Additional active vitamin D compounds having a reduced hypercalcemic effect include Ro23-7553 and Ro24-5531 available from Hoffman LaRoche. Other examples of active vitamin D compounds having a reduced hypercalcemic effect can be found in U.S. Pat. No. 4,717,721. Determining the hypercalcemic effect of an active vitamin D compound is routine in the art and can be carried out as disclosed in Hansen et al., Curr. Pharm. Des. 6:803-828 (2000).

The pharmaceutical compositions of the present invention may further comprise one or more additives. Additives that are well known in the art include, e.g., detackifiers, anti-foaming agents, buffering agents, antioxidants (e.g., ascorbic acid, ascorbyl palmitate, sodium ascorbate, butylated hydroxyanisole (BHA), butylated hydroxytoluene (BHT), propyl gallate, malic acid, fumaric acid, potassium metabisulfite, sodium bisulfite, sodium metabisulfite, and tocopherols, e.g., α-tocopherol (vitamin E)), preservatives, chelating agents, viscomodulators, tonicifiers, flavorants, colorants, odorants, opacifiers, suspending agents, binders, fillers, plasticizers, lubricants, and mixtures thereof. The amounts of such additives can be readily determined by one skilled in the art, according to the particular properties desired. For example, antioxidants may be present in an amount of from about 0.01% to about 0.5% by weight based upon the total weight of the composition, preferably about 0.05% to about 0.35%.

The additive may also comprise a thickening agent. Suitable thickening agents may be of those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents as aforesaid will generally not be required and is generally less preferred. Use of thickening agents is, on the other hand, indicated, e.g., where topical application is foreseen.

Compositions in accordance with the present invention may be employed for administration in any appropriate manner, e.g., orally, e.g., in unit dosage form, for example in a solution, in hard or soft encapsulated form including gelatin encapsulated form. Gelatin capsules may be sealed by banding or liquid microspray sealing. Compositions may also be administered parenterally or topically, e.g., for application to the skin, for example in the form of a cream, paste, lotion, gel, ointment, poultice, cataplasm, plaster, dermal patch or the like, or for ophthalmic application, for example in the form of an eye-drop, -lotion or -gel formulation. Readily flowable forms, for example solutions and emulsions, may also be employed e.g., for intralesional injection, or may be administered rectally, e.g., as an enema. The compositions may additionally contain agents that enhance the delivery of the active vitamin D compound, e.g., liposomes, polymers or co-polymers (e.g., branched chain polymers).

When the composition of the present invention is formulated in unit dosage form, the active vitamin D compound will preferably be present in an amount of between 1 and 400 μg per unit dose. More preferably, the amount of active vitamin D compound per unit dose will be about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 305, 310, 315, 320, 325, 330, 335, 340, 345, 350, 355, 360, 365, 370, 375, 380, 385, 390, 395, or 400 μg or any amount therein. In a preferred embodiment, the amount of active vitamin D compound per unit dose will be about 5 μg to about 180 μg, more preferably about 10 μg to about 135 μg, more preferably about 45 μg. In one embodiment, the unit dosage form comprises 45, 90, 135, or 180 μg of calcitriol.

When the unit dosage form of the composition is a capsule, the total quantity of ingredients present in the capsule is preferably about 10-1000 μL. More preferably, the total quantity of ingredients present in the capsule is about 100-300 μL. In another embodiment, the total quantity of ingredients present in the capsule is preferably about 10-1500 mg, preferably about 100-1000 mg. In one embodiment, the total quantity is about 225, 450, 675, or 900 mg. In one embodiment, the unit dosage form is a capsule comprising 45, 90, 135, or 180 μg of calcitriol.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative of preferred or individually inventive teachings only and not as not limiting the invention in its broadest aspect.

The lipophilic phase component of the invention will suitably be present in an amount of from about 10% to about 90% by weight based upon the total weight of the composition. Preferably, the lipophilic phase component is present in an amount of from about 15% to about 65% by weight based upon the total weight of the composition.

The surfactant or surfactants of the invention will suitably be present in an amount of from about 1% to 90% by weight based upon the total weight of the composition. Preferably, the surfactant(s) is present in an amount of from about 5% to about 85% by weight based upon the total weight of the composition.

The amount of active vitamin D compound in compositions of the invention will of course vary, e.g., depending on the intended route of administration and to what extent other components are present. In general, however, the active vitamin D compound of the invention will suitably be present in an amount of from about 0.005% to 20% by weight based upon the total weight of the composition. Preferably, the active vitamin D compound is present in an amount of from about 0.01% to 15% by weight based upon the total weight of the composition.

The hydrophilic phase component of the invention will suitably be present in an amount of from about 2% to about 20% by weight based upon the total weight of the composition. Preferably, the hydrophilic phase component is present in an amount of from about 5% to 15% by weight based upon the total weight of the composition.

The pharmaceutical composition of the invention may be in a semisolid formulation. Semisolid formulations within the scope of the invention may comprise, e.g., a lipophilic phase component present in an amount of from about 50% to about 80% by weight based upon the total weight of the composition, a surfactant present in an amount of from about 5% to about 50% by weight based upon the total weight of the composition, and an active vitamin D compound present in an amount of from about 0.01% to about 15% by weight based upon the total weight of the composition.

The pharmaceutical compositions of the invention may be in a liquid formulation. Liquid formulations within the scope of the invention may comprise, e.g., a lipophilic phase component present in an amount of from about 50% to about 60% by weight based upon the total weight of the composition, a surfactant present in an amount of from about 4% to about 25% by weight based upon the total weight of the composition, an active vitamin D compound present in an amount of from about 0.01% to about 15% by weight based upon the total weight of the composition, and a hydrophilic phase component present in an amount of from about 5% to about 10% by weight based upon the total weight of the composition.

Additional compositions that may be used include the following, wherein the percentage of each component is by weight based upon the total weight of the composition excluding the active vitamin D compound:

| | | |
|---|---|---|
| a. | Gelucire 44/14 | about 50% |
| | Miglyol 812 | about 50%; |
| b. | Gelucire 44/14 | about 50% |
| | Vitamin E TPGS | about 10% |
| | Miglyol 812 | about 40%; |
| c. | Gelucire 44/14 | about 50% |
| | Vitamin E TPGS | about 20% |
| | Miglyol 812 | about 30%; |
| d. | Gelucire 44/14 | about 40% |
| | Vitamin E TPGS | about 30% |
| | Miglyol 812 | about 30%; |
| e. | Gelucire 44/14 | about 40% |
| | Vitamin E TPGS | about 20% |
| | Miglyol 812 | about 40%; |
| f. | Gelucire 44/14 | about 30% |
| | Vitamin E TPGS | about 30% |
| | Miglyol 812 | about 40%; |
| g. | Gelucire 44/14 | about 20% |
| | Vitamin E TPGS | about 30% |
| | Miglyol 812 | about 50%; |
| h. | Vitamin E TPGS | about 50% |
| | Miglyol 812 | about 50%; |
| i. | Gelucire 44/14 | about 60% |
| | Vitamin E TPGS | about 25% |
| | Miglyol 812 | about 15%; |
| j. | Gelucire 50/13 | about 30% |
| | Vitamin E TPGS | about 5% |
| | Miglyol 812 | about 65%; |
| k. | Gelucire 50/13 | about 50% |
| | Miglyol 812 | about 50%; |
| l. | Gelucire 50/13 | about 50% |
| | Vitamin E TPGS | about 10% |
| | Miglyol 812 | about 40%; |
| m. | Gelucire 50/13 | about 50% |
| | Vitamin E TPGS | about 20% |
| | Miglyol 812 | about 30%; |
| n. | Gelucire 50/13 | about 40% |
| | Vitamin E TPGS | about 30% |
| | Miglyol 812 | about 30%; |
| o. | Gelucire 50/13 | about 40% |
| | Vitamin E TPGS | about 20% |
| | Miglyol 812 | about 40%; |
| p. | Gelucire 50/13 | about 30% |
| | Vitamin E TPGS | about 30% |
| | Miglyol 812 | about 40%; |
| q. | Gelucire 50/13 | about 20% |
| | Vitamin E TPGS | about 30% |
| | Miglyol 812 | about 50%; |
| r. | Gelucire 50/13 | about 60% |
| | Vitamin E TPGS | about 25% |
| | Miglyol 812 | about 15%; |
| s. | Gelucire 44/14 | about 50% |
| | PEG 4000 | about 50%; |
| t. | Gelucire 50/13 | about 50% |
| | PEG 4000 | about 50%; |
| u. | Vitamin E TPGS | about 50% |
| | PEG 4000 | about 40%; |
| v. | Gelucire 44/14 | about 33.3% |
| | Vitamin E TPGS | about 33.3% |
| | PEG 4000 | about 33.3%; |
| w. | Gelucire 50/13 | about 33.3% |
| | Vitamin E TPGS | about 33.3% |
| | PEG 4000 | about 33.3%; |
| x. | Gelucire 44/14 | about 50% |
| | Vitamin E TPGS | about 50%; |
| y. | Gelucire 50/13 | about 50% |
| | Vitamin E TPGS | about 50%; |
| z. | Vitamin E TPGS | about 5% |
| | Miglyol 812 | about 95%; |
| aa. | Vitamin E TPGS | about 5% |
| | Miglyol 812 | about 65% |
| | PEG 4000 | about 30%; |
| ab. | Vitamin E TPGS | about 10% |
| | Miglyol 812 | about 90%; |
| ac. | Vitamin E TPGS | about 5% |
| | Miglyol 812 | about 85% |
| | PEG 4000 | about 10%; and |
| ad. | Vitamin E TPGS | about 10% |
| | Miglyol 812 | about 80% |
| | PEG 4000 | about 10%. |

In one embodiment of the invention, the pharmaceutical compositions comprise an active vitamin D compound, a lipophilic component, and a surfactant. The lipophilic component may be present in any percentage from about 1% to about 100%. The lipophilic component may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant may be present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In preferred embodiments, the pharmaceutical compositions comprise about 50% MIGLYOL 812 and about 50% vitamin E TPGS, about 90% MIGLYOL 812 and about 10% vitamin E TPGS, or about 95% MIGLYOL 812 and about 5% vitamin E TPGS.

In another embodiment of the invention, the pharmaceutical compositions comprise an active vitamin D compound and a lipophilic component, e.g., around 100% MIGLYOL 812.

In a preferred embodiment, the pharmaceutical compositions comprise about 50% MIGLYOL 812, about 50% vitamin E TPGS, and small amounts of BHA and BHT. This formulation has been shown to be unexpectedly stable, both chemically and physically (see Example 16). In a particularly preferred embodiment, the pharmaceutical compositions comprise about 50% MIGLYOL 812, about 50% vitamin E TPGS, and about 0.35% each of BHA and BHT. The enhanced stability provides the compositions with a longer shelf life. Importantly, the stability also allows the compositions to be stored at room temperature, thereby avoiding the complication and cost of storage under refrigeration. Additionally, this composition is suitable for oral administration and has been shown to be capable of solubilizing high doses of active vitamin D compound, thereby enabling high dose pulse administration of active vitamin D compounds for the treatment of hyperproliferative diseases and other disorders.

In addition to the foregoing the present invention also provides a process for the production of a pharmaceutical composition as hereinbefore defined, which process comprises bringing the individual components thereof into intimate admixture and, when required, compounding the obtained composition in unit dosage form, for example filling said composition into gelatin, e.g., soft or hard gelatin, capsules, or non-gelatin capsules.

In a more particular embodiment, the invention provides a process for the preparation of a pharmaceutical composition, which process comprises bringing an active vitamin D compound, e.g., calcitriol, into close admixture with a lipophilic phase component and a surfactant as hereinbefore defined, the relative proportion of the lipophilic phase component and the surfactant being selected relative to the quantity of active vitamin D compound employed, such that an emulsion pre-concentrate is obtained.

The present invention also provides methods for the treatment and prevention of hyperproliferative diseases such as cancer and psoriasis, said methods comprising administering an active vitamin D compound in an emulsion pre-concentrate formulation to a patient in need thereof. Alternatively, the active vitamin D compound can be administered in an emulsion formulation that is made by diluting an emulsion pre-concentrate of the present invention with an appropriate quantity of water. Alternatively, the active vitamin D compound can be administered in any formulation disclosed herein.

The term “cancer,” as used herein, is intended to refer to any known cancer, and may include, but is not limited to the following: leukemias such as acute leukemia, acute lymphocytic leukemia, acute myelocytic leukemias such as myeloblastic, promyelocytic, myelomonocytic, monocytic, and erythroleukemia leukemias, and myelodysplastic syndrome; chronic leukemias such as chronic myelocytic (granulocytic) leukemia, chronic lymphocytic leukemia, and hairy cell leukemia; polycythemia vera; lymphomas such as Hodgkin's disease and non-Hodgkin's disease; multiple myelomas such as smoldering multiple myeloma, non-secretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancers such as adenocarcinoma, lobular (small cell) carcinoma, intraductal carcinoma, medullary breast cancer, mucinous breast cancer, tubular breast cancer, papillary breast cancer, Paget's disease of the breast, and inflammatory breast cancer; adrenal cancers such as pheochromocytoma and adrenocortical carcinoma; thyroid cancers such as papillary or follicular thyroid cancer, medullary thyroid cancer and anaplastic thyroid cancer; pancreatic cancers such as insulinoma, gastrinoma, glucagonoma, vipoma, somatostatin-secreting tumor, and carcinoid or islet cell tumor; pituitary cancers such as prolactin-secreting tumor and acromegaly; eye cancers such as ocular melanoma, iris melanoma, choroidal melanoma, and cilliary body melanoma, and retinoblastoma; vaginal cancers such as squamous cell carcinoma, adenocarcinoma, and melanoma; vulvar cancers such as squamous cell carcinoma, melanoma, adenocarcinoma, basal cell carcinoma, sarcoma, and Paget's disease of the genitals; cervical cancers such as squamous cell carcinoma and adenocarcinoma; uterine cancers such as endometrial carcinoma and uterine sarcoma; ovarian cancers such as ovarian epithelial carcinoma, ovarian epithelial borderline tumor, germ cell tumor, and stromal tumor; esophageal cancers such as squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as adenocarcinoma, fingating (polypoid), ulcerating, superficial spreading, diffusely spreading, malignant lymphoma, liposarcoma, fibrosarcoma, and carcinosarcoma; colon cancers; rectal cancers; liver cancers such as hepatocellular carcinoma and hepatoblastoma, gallbladder cancers such as adenocarcinoma; cholangiocarcinomas such as papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as germinal tumor, seminoma, anaplastic, classic (typical), spermatocytic, nonseminoma, embryonal carcinoma, teratoma carcinoma, and choriocarcinoma (yolk-sac tumor), prostate cancers such as adenocarcinoma, leiomyosarcoma, and rhabdomyosarcoma; penile cancers; oral cancers such as squamous cell carcinoma; basal cancers; salivary gland cancers such as adenocarcinoma, mucoepidermoid carcinoma, and adenoidcystic carcinoma; pharynx cancers such as squamous cell cancer and verrucous; skin cancers such as basal cell carcinoma, squamous cell carcinoma and melanoma, superficial spreading melanoma, nodular melanoma, lentigo malignant melanoma, acral lentiginous melanoma; head and neck cancers; kidney cancers such as renal cell cancer, adenocarcinoma, hypemephroma, fibrosarcoma, transitional cell cancer (renal pelvis and/or ureter); Wilms' tumor; and bladder cancers such as transitional cell carcinoma, squamous cell cancer, adenocarcinoma, and carcinosarcoma. In addition, cancers that can be treated by the methods and compositions of the present invention include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinoma. See Fishman et al., 1985, Medicine, 2d Ed., J.B. Lippincott Co., Philadelphia, Pa. and Murphy et al., 1997, Informed Decisions: The Complete Book of Cancer Diagnosis, Treatment, and Recovery, Viking Penguin, New York, N.Y., for a review of such disorders.

The active vitamin D compound is preferably administered at a dose of about 1 μg to about 400 μg, more preferably from about 15 μg to about 300 μg. In a specific embodiment, an effective amount of an active vitamin D compound is 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 305, 310, 315, 320, 325, 330, 335, 340, 345, 350, 355, 360, 365, 370, 375, 380, 385, 390, 395, or 400 μg or more. In certain embodiments, an effective dose of an active vitamin D compound is between about 1 μg to about 270 μg, more preferably between about 15 μg to about 225 μg, more preferably between about 15 μg to about 180 μg, more preferably between about 15 μg to about 135 μg, more preferably between about 20 μg to about 90 μg, more preferably between about 30 μg to about 60 μg, and even more preferably about 45 μg. In certain embodiments, the methods of the invention comprise administering an active vitamin D compound in a dose of about 0.12 μg/kg bodyweight to about 3 μg/kg bodyweight. The compound may be administered by any route, including oral, intramuscular, intravenous, parenteral, rectal, nasal, topical, or transdermal.

If the compound is to be administered daily, the dose may be kept low, for example about 0.5 μg to about 5 μg, in order to avoid or diminish the induction of hypercalcemia. If the active vitamin D compound has a reduced hypercalcemic effect a higher daily dose may be administered without resulting in hypercalcemia, for example about 10 μg to about 20 μg or higher (up to about 50 μg to about 100 μg).

In a preferred embodiment of the invention, the active vitamin D compound is administered in a pulsed-dose fashion so that high doses of the active vitamin D compound can be administered without inducing hypercalcemia. Pulsed dosing refers to intermittently administering an active vitamin D compound on either a continuous intermittent dosing schedule or a non-continuous intermittent dosing schedule. High doses of active vitamin D compounds include doses greater than about 3 μg as discussed in the sections above. Therefore, in certain embodiments of the invention, the methods for the treatment or amelioration of cancer encompass intermittently administering high doses of active vitamin D compounds. The frequency of the pulsed-dose administration can be limited by a number of factors including but not limited to the pharmacokinetic parameters of the compound or formulation and the pharmacodynamic effects of the active vitamin D compound on the animal. For example, animals with cancer having impaired renal function may require less frequent administration of the active vitamin D compound because of the decreased ability of those animals to excrete calcium.

The following is exemplary only and merely serves to illustrate that the term “pulsed-dose” can encompass any discontinuous administration regimen designed by a person of skill in the art.

In one example, the active vitamin D compound can be administered not more than once every three days, every four days, every five days, every six days, every seven days, every eight days, every nine days, ten days, every two weeks, every three weeks, every four weeks, every five weeks, every six weeks, every seven weeks, every eight weeks, or longer. The administration can continue for one, two, three, or four weeks or one, two, or three months, or longer. Optionally, after a period of rest, the active vitamin D compound can be administered under the same or a different schedule. The period of rest can be one, two, three, or four weeks, or longer, according to the pharmacodynamic effects of the active vitamin D compound on the animal.

In another example, the active vitamin D compound can be administered once per week for three months.

In a preferred embodiment, the active vitamin D compound can be administered once per week for three weeks of a four week cycle. After a one week period of rest, the active vitamin D compound can be administered under the same or different schedule.

In another preferred embodiment the active vitamin D compound an be administered once every three weeks.

Further examples of dosing schedules that can be used in the methods of the present invention are provided in U.S. Pat. No. 6,521,608, which is incorporated by reference in its entirety.

The above-described administration schedules are provided for illustrative purposes only and should not be considered limiting. A person of skill in the art will readily understand that all active vitamin D compounds are within the scope of the invention and that the exact dosing and schedule of administration of the active vitamin D compounds can vary due to many factors.

The amount of a therapeutically effective dose of a pharmaceutical agent in the acute or chronic management of a disease or disorder may differ depending on factors including but not limited to the disease or disorder treated, the specific pharmaceutical agents and the route of administration. According to the methods of the invention, an effective dose of an active vitamin D compound is any dose of the compound effective to treat or ameliorate cancer or other hyperproliferative diseases. A high dose of an active vitamin D compound can be a dose from about 3 μg to about 400 μg or any dose within this range as discussed above. The dose, dose frequency, duration, or any combination thereof, may also vary according to age, body weight, response, and the past medical history of the animal as well as the route of administration, pharmacokinetics, and pharmacodynamic effects of the pharmaceutical agents. These factors are routinely considered by one of skill in the art.

The rates of absorption and clearance of vitamin D compounds are affected by a variety of factors that are well known to persons of skill in the art. As discussed above, the pharmacokinetic properties of active vitamin D compounds limit the peak concentration of vitamin D compounds that can be obtained in the blood without inducing the onset of hypercalcemia. The rate and extent of absorption, distribution, binding or localization in tissues, biotransformation, and excretion of the active vitamin D compound can all affect the frequency at which the pharmaceutical agents can be administered. In certain embodiments, active vitamin D compounds are administered in a pulsed-dose fashion in high doses as a method of treating or ameliorating cancer according to the dosing schedule described above.

In one embodiment of the invention, an active vitamin D compound is administered at a dose sufficient to achieve peak plasma concentrations of the active vitamin D compound of about 0.1 nM to about 20 nM. In certain embodiments, the methods of the invention comprise administering the active vitamin D compound in a dose that achieves peak plasma concentrations of 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 12.5 nM, 15 nM, 17.5 nM or 20 nM, or any range of concentrations therein. In other embodiments, the active vitamin D compound is administered in a dose that achieves peak plasma concentrations of the active vitamin D compound exceeding about 0.5 nM, preferably about 0.5 nM to about 20 nM, more preferably about 1 nM to about 10 nM, more preferably about 1 nM to about 7 nM, and even more preferably about 3 nM to about 5 nM.

In another preferred embodiment, the active vitamin D compound is administered at a dose of at least about 0.12 μg/kg bodyweight, more preferably at a dose of at least about 0.5 μg/kg bodyweight.

One of skill in the art will recognize that these standard doses are for an average sized adult of approximately 70 kg and can be adjusted for the factors routinely considered as stated above.

In certain embodiments, the methods of the invention further comprise administering a dose of an active vitamin D compound that achieves peak plasma concentrations rapidly, e.g., within four hours. In further embodiments, the methods of the invention comprise administering a dose of an active vitamin D compound that is eliminated quickly, e.g., with an elimination half-life of less than 12 hours.

While obtaining high concentrations of the active vitamin D compound is beneficial, it must be balanced with clinical safety, e.g., hypercalcemia. Thus, in one aspect of the invention, the methods of the invention encompass intermittently administering high doses of active vitamin D compounds to a subject with cancer or another hyperproliferative disease and monitoring the subject for symptoms associated with hypercalcemia. Such symptoms include calcification of soft tissues (e.g., cardiac tissue), increased bone density, and hypercalcemic nephropathy. In still another embodiment, the methods of the invention encompass intermittently administering high doses of an active vitamin D compound to a subject with cancer or another hyperproliferative disease and monitoring the calcium plasma concentration of the subject to ensure that the calcium plasma concentration is less than about 10.2 mg/dL.

In certain embodiments, high blood levels of vitamin D compounds can be safely obtained in conjunction with reducing the transport of calcium into the blood. In one embodiment, higher active vitamin D compound concentrations are safely obtainable without the onset of hypercalcemia when administered in conjunction with a reduced calcium diet. In one example, the calcium can be trapped by an adsorbent, absorbent, ligand, chelate, or other binding moiety that cannot be transported into the blood through the small intestine. In another example, the rate of osteoclast activation can be inhibited by administering, for example, a bisphosphonate such as, e.g., zoledronate, pamidronate, or alendronate, or a glucocorticoid, such as, e.g., prednisone or dexamethasone, in conjunction with the active vitamin D compound.

In certain embodiments, high blood levels of active vitamin D compounds are safely obtained in conjunction with maximizing the rate of clearance of calcium. In one example, calcium excretion can be increased by ensuring adequate hydration and salt intake. In another example, diuretic therapy can be used to increase calcium excretion.

In certain embodiments of the invention, the methods for the treatment and prevention of hyperproliferative diseases such as cancer and psoriasis further comprise the administration of a chemotherapeutic agent or radiotherapeutic agent or treatment along with the active vitamin D compound.

The term “chemotherapeutic agent,” as used herein, is intended to refer to any chemotherapeutic agent known to those of skill in the art to be effective for the treatment or amelioration of cancer. Chemotherapeutic agents include, but are not limited to; small molecules; synthetic drugs; peptides; polypeptides; proteins; nucleic acids (e.g., DNA and RNA polynucleotides including, but not limited to, antisense nucleotide sequences, triple helices and nucleotide sequences encoding biologically active proteins, polypeptides or peptides); antibodies; synthetic or natural inorganic molecules; mimetic agents; and synthetic or natural organic molecules. Any agent which is known to be useful, or which has been used or is currently being used for the treatment or amelioration of cancer can be used in combination with an active vitamin D compound in accordance with the invention described herein. See, e.g., Hardman et al., eds., 1996, Goodman & Gilman's The Pharmacological Basis Of Therapeutics 9th Ed, Mc-Graw-Hill, New York, N.Y. for information regarding therapeutic agents which have been or are currently being used for the treatment or amelioration of cancer.

Chemotherapeutic agents useful in the methods and compositions of the invention include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, and angiogenesis inhibitors.

Other chemotherapeutic agents that may be used include abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

Chemotherapeutic agents may be administered at doses that are recognized by those of skill in the art to be effective for the treatment of pancreatic cancer. In certain embodiments, chemotherapeutic agents may be administered at doses lower than those used in the art due to the additive or synergistic effect of the active vitamin D compound.

The term “radiotherapeutic agent,” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy.

Brachytherapy can be administered according to any schedule, dose, or method known to one of skill in the art to be effective in the treatment or amelioration of cancer, without limitation. In general, brachytherapy comprises insertion of radioactive sources into the body of a subject to be treated for cancer, preferably inside the tumor itself, such that the tumor is maximally exposed to the radioactive source, while preferably minimizing the exposure of healthy tissue. Representative radioisotopes that can be administered in brachytherapy include, but are not limited to, phosphorus 32, cobalt 60, palladium 103, ruthenium 106, iodine 125, cesium 137, iridium 192, xenon 133, radium 226, californium 252, or gold 198. Methods of administering and apparatuses and compositions useful for brachytherapy are described in Mazeron et al., Sem. Rad. Onc. 12:95-108 (2002) and U.S. Pat. Nos. 6,319,189, 6,179,766, 6,168,777, 6,149,889, and 5,611,767.

Radionuclide therapy can be administered according to any schedule, dose, or method known to one of skill in the art to be effective in the treatment or amelioration of cancer, without limitation. In general, radionuclide therapy comprises systemic administration of a radioisotope that preferentially accumulates in or binds to the surface of cancerous cells. The preferential accumulation of the radionuclide can be mediated by a number of mechanisms, including, but not limited to, incorporation of the radionuclide into rapidly proliferating cells, specific accumulation of the radionuclide by the cancerous tissue without special targeting, or conjugation of the radionuclide to a biomolecule specific for a neoplasm.

Representative radioisotopes that can be administered in radionuclide therapy include, but are not limited to, phosphorus 32, yttrium 90, dysprosium 165, indium 111, strontium 89, samarium 153, rhenium 186, iodine 131, iodine 125, lutetium 177, and bismuth 213. While all of these radioisotopes may be linked to a biomolecule providing specificity of targeting, iodine 131, indium 111, phosphorus 32, samarium 153, and rhenium 186 may be administered systemically without such conjugation. One of skill in the art may select a specific biomolecule for use in targeting a particular neoplasm for radionuclide therapy based upon the cell-surface molecules present on that neoplasm. Examples of biomolecules providing specificity for particular cell are reviewed in an article by Thomas, Cancer Biother. Radiopharm. 17:71-82 (2002), which is incorporated herein by reference in its entirety. Furthermore, methods of administering and compositions useful for radionuclide therapy may be found in U.S. Pat. Nos. 6,426,400, 6,358,194, 5,766,571.

The term “radiotherapeutic treatment,” as used herein, is intended to refer to any radiotherapeutic treatment known to one of skill in the art to be effective to treat or ameliorate cancer, without limitation. For instance, the radiotherapeutic treatment can be external-beam radiation therapy, thermotherapy, radiosurgery, charged-particle radiotherapy, neutron radiotherapy, or photodynamic therapy.

External-beam radiation therapy can be administered according to any schedule, dose, or method known to one of skill in the art to be effective in the treatment or amelioration of cancer, without limitation. In general, external-beam radiation therapy comprises irradiating a defined volume within a subject with a high energy beam, thereby causing cell death within that volume. The irradiated volume preferably contains the entire cancer to be treated, and preferably contains as little healthy tissue as possible. Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652.

Thermotherapy can be administered according to any schedule, dose, or method known to one of skill in the art to be effective in the treatment or amelioration of cancer, without limitation. In certain embodiments, the thermotherapy can be cryoablation therapy. In other embodiments, the thermotherapy can be hyperthermic therapy. In still other embodiments, the thermotherapy can be a therapy that elevates the temperature of the tumor higher than in hyperthermic therapy.

Cryoablation therapy involves freezing of a neoplastic mass, leading to deposition of intra- and extra-cellular ice crystals; disruption of cellular membranes, proteins, and organelles; and induction of a hyperosmotic environment, thereby causing cell death. Methods for and apparatuses useful in cryoablation therapy are described in Murphy et al., Sem. Urol. Oncol. 19:133-140 (2001) and U.S. Pat. Nos. 6,383,181, 6,383,180, 5,993,444, 5,654,279, 5,437,673, and 5,147,355.

Hyperthermic therapy typically involves elevating the temperature of a neoplastic mass to a range from about 42° C. to about 44° C. The temperature of the cancer may be further elevated above this range; however, such temperatures can increase injury to surrounding healthy tissue while not causing increased cell death within the tumor to be treated. The tumor may be heated in hyperthermic therapy by any means known to one of skill in the art without limitation. For example, and not by way of limitation, the tumor may be heated by microwaves, high intensity focused ultrasound, ferromagnetic thermoseeds, localized current fields, infrared radiation, wet or dry radiofrequency ablation, laser photocoagulation, laser interstitial thermic therapy, and electrocautery. Microwaves and radiowaves can be generated by waveguide applicators, horn, spiral, current sheet, and compact applicators.

Other methods of and apparatuses and compositions for raising the temperature of a tumor are reviewed in an article by Wust et al., Lancet Oncol. 3:487-97 (2002), and described in U.S. Pat. Nos. 6,470,217, 6,379,347, 6,165,440, 6,163,726, 6,099,554, 6,009,351, 5,776,175, 5,707,401, 5,658,234, 5,620,479, 5,549,639, and 5,523,058.

Radiosurgery can be administered according to any schedule, dose, or method known to one of skill in the art to be effective in the treatment or amelioration of cancer, without limitation. In general, radiosurgery comprises exposing a defined volume within a subject to a manually directed radioactive source, thereby causing cell death within that volume. The irradiated volume preferably contains the entire cancer to be treated, and preferably contains as little healthy tissue as possible. Typically, the tissue to be treated is first exposed using conventional surgical techniques, then the radioactive source is manually directed to that area by a surgeon. Alternatively, the radioactive source can be placed near the tissue to be irradiated using, for example, a laparoscope. Methods and apparatuses useful for radiosurgery are further described in Valentini et al., Eur. J. Surg. Oncol. 28:180-185 (2002) and in U.S. Pat. Nos. 6,421,416, 6,248,056, and 5,547,454.

Charged-particle radiotherapy can be administered according to any schedule, dose, or method known to one of skill in the art to be effective in the treatment or amelioration of cancer, without limitation. In certain embodiments, the charged-particle radiotherapy can be proton beam radiotherapy. In other embodiments, the charged-particle radiotherapy can be helium ion radiotherapy. In general, charged-particle radiotherapy comprises irradiating a defined volume within a subject with a charged-particle beam, thereby causing cellular death within that volume. The irradiated volume preferably contains the entire cancer to be treated, and preferably contains as little healthy tissue as possible. A method for administering charged-particle radiotherapy is described in U.S. Pat. No. 5,668,371.

Neutron radiotherapy can be administered according to any schedule, dose, or method known to one of skill in the art to be effective in the treatment or amelioration of cancer, without limitation. In certain embodiments, the neutron radiotherapy can be a neutron capture therapy. In such embodiments, a compound that emits radiation when bombarded with neutrons and preferentially accumulates in a neoplastic mass is administered to a subject. Subsequently, the tumor is irradiated with a low energy neutron beam, activating the compound and causing it to emit decay products that kill the cancerous cells. The compound to be activated can be caused to preferentially accumulate in the target tissue according to any of the methods useful for targeting of radionuclides, as described above, or in the methods described in Laramore, Semin. Oncol. 24:672-685 (1997) and in U.S. Pat. Nos. 6,400,796, 5,877,165, 5,872,107, and 5,653,957.

In other embodiments, the neutron radiotherapy can be a fast neutron radiotherapy. In general, fast neutron radiotherapy comprises irradiating a defined volume within a subject with a neutron beam, thereby causing cellular death within that volume.

Photodynamic therapy can be administered according to any schedule, dose, or method known to one of skill in the art to be effective in the treatment or amelioration of cancer, without limitation. In general, photodynamic therapy comprises administering a photosensitizing agent that preferentially accumulates in a neoplastic mass and sensitizes the neoplasm to light, then exposing the tumor to light of an appropriate wavelength. Upon such exposure, the photosensitizing agent catalyzes the production of a cytotoxic agent, such as, e.g., singlet oxygen, which kills the cancerous cells. Methods of administering and apparatuses and compositions useful for photodynamic therapy are disclosed in Hopper, Lancet Oncol. 1:212-219 (2000) and U.S. Pat. Nos. 6,283,957, 6,071,908, 6,011,563, 5,855,595, 5,716,595, and 5,707,401.

While not intending to be bound by any particular theory of operation, it is believed that active vitamin D compounds can enhance the sensitivity of cancerous cells to radiotherapy, and this enhanced sensitivity is due to changes in cell mechanisms regulating apoptosis and/or the cell cycle. Administration of an active vitamin D compound can not only enhance but also expand the applicability of radiotherapy in the treatment or amelioration of cancer, that would otherwise not respond to current radiotherapy. Further, sensitizing cells to treatment can allow use of a lower dose of radiotherapy, which reduces the side effects associated with the radiotherapy.

Radiotherapy can be administered to destroy tumor cells before or after surgery, before or after chemotherapy, and sometimes during chemotherapy. Radiotherapy may also be administered for palliative reasons to relieve symptoms of cancer, for example, to lessen pain. Among the types of tumors that can be treated using radiotherapy are localized tumors that cannot be excised completely and metastases and tumors whose complete excision would cause unacceptable functional or cosmetic defects or be associated with unacceptable surgical risks.

It will be appreciated that both the particular radiation dose to be utilized in treating cancer and the method of administration will depend on a variety of factors. Thus, the dosages of radiation that can be used according to the methods of the present invention are determined by the particular requirements of each situation. The dosage will depend on such factors as the size of the tumor, the location of the tumor, the age and sex of the patient, the frequency of the dosage, the presence of other tumors, possible metastases and the like. Those skilled in the art of radiotherapy can readily ascertain the dosage and the method of administration for any particular tumor by reference to Hall, E. J., Radiobiology for the Radiobiologist, 5th edition, Lippincott Williams & Wilkins Publishers, Philadelphia, Pa., 2000; Gunderson, L. L. and Tepper J. E., eds., Clinical Radiation Oncology, Churchill Livingstone, London, England, 2000; and Grosch, D. S., Biological Effects of Radiation, 2nd edition, Academic Press, San Francisco, Calif., 1980. In certain embodiments, radiotherapeutic agents and treatments may be administered at doses lower than those known in the art due to the additive or synergistic effect of the active vitamin D compound.

The dosage amounts and frequencies of administration of the additional therapeutic agents provided herein are encompassed by the terms therapeutically effective. The dosage and frequency of these agents further will typically vary according to factors specific for each patient depending on the specific therapeutic agents administered, the severity and type of pancreatic cancer, the route of administration, as well as age, body weight, response and the past medical history of the patient. Suitable regimens can be selected by one skilled in the art by considering such factors and by following, for example, dosages reported in the literature and recommended in the Physician's Desk Reference (56^{th} ed., 2002).

For animals that have resectable cancer, the active vitamin D compound can be administered prior to and/or after surgery. Similarly, the chemotherapeutic agents and radiotherapeutic agents or treatments can be administered prior to and/or after surgery.

Any period of treatment with the active vitamin D compound prior to, during or after the administration of the chemotherapeutic agents or radiotherapeutic agents or treatments can be employed in the present invention. The exact period for treatment with the active vitamin D compound will vary depending upon the active vitamin D compound used, the type of pancreatic cancer, the patient, and other related factors. The active vitamin D compound may be administered as little as 12 hours and as much as 3 months prior to or after the administration of the chemotherapeutic agents or radiotherapeutic agents or treatments. The active vitamin D may be administered at least one day before or after administration of the chemotherapeutic agents or radiotherapeutic agents or treatments and for as long as 3 months before or after administration of the chemotherapeutic agents or radiotherapeutic agents or treatments. In certain embodiments, the methods of the invention comprise administering the active vitamin D compound once every 3, 4, 5, 6, 7, 8, 9, or 10 days for a period of 3 days to 60 days before or after administration of the chemotherapeutic agents or radiotherapeutic agents or treatments.

The administration of the active vitamin D compound may be continued concurrently with the administration of the chemotherapeutic agents or radiotherapeutic agents or treatments. Additionally, the administration of the active vitamin D compound may be continued beyond the administration of the chemotherapeutic agents or radiotherapeutic agents or treatments.

In certain embodiments of the invention, the method of administering an active vitamin D compound alone or in combination with chemotherapeutic agents or radiotherapeutic agents or treatments may be repeated at least once. The method my be repeated as many times as necessary to achieve or maintain a therapeutic response, e.g., from one to about ten times. With each repetition of the method the active vitamin D compound and the chemotherapeutic agents or radiotherapeutic agents or treatments may be the same or different from that used in the previous repetition. Additionally, the time period of administration of the active vitamin D compound and the manner in which it is administered can vary from repetition to repetition.

In a preferred embodiment, the cancers are treated by combination chemotherapy as disclosed in U.S. Pat. Nos. 6,087,350 and 6,559,139. In this embodiment, active vitamin D compounds are administered in combination with other pharmaceutical agents, in particular cytotoxic agents for the treatment of hyperproliferative disease. Preferably, the pretreatment of hyperproliferative cells with active vitamin D compounds followed by treatment with cytotoxic agents enhances the efficacy of the cytotoxic agents. For example, the active vitamin D compound may be administered one day before the chemotherapeutic agent.

Animals which may be treated according to the present invention include all animals which may benefit from administration of the formulations of the present invention. Such animals include humans, pets such as dogs and cats, and veterinary animals such as cows, pigs, sheep, goats and the like.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Relative Chemical Compatibility of Calcitriol with Selected Components

In this example, the relative chemical compatibility of calcitriol with selected lipophilic, hydrophilic and surfactant components was evaluated by measuring the percent recovery of intact calcitriol after storage at 40° C. and 60° C. Calcitriol recovery was determined based on analyses of high-pressure liquid chromatography (HPLC). The results are presented in Table 1.

TABLE 1
Percent Recovery of Calcitriol Formulated
in Selected Components

| | | | | % | % |
|---|---|---|---|---|---|
| | | | | Recovery | Recovery |
| | | | | at | at |
| Component | Excipient | Time | | 40° C. | 60° C. |
| Lipophilic | Corn oil | 0 | | 100.00 | 100.00 |
| | | 3 | days | 93.77 | 104.80 |
| | | 7 | days | 90.27 | 91.50 |
| | | 14 | days | 89.89 | 86.46 |
| | Soybean | 0 | | 100.00 | 100.00 |
| | oil | 3 | days | 96.44 | 94.56 |
| | | 7 | days | 98.46 | 98.57 |
| | | 14 | days | 96.66 | 93.15 |
| | Sunflower | 0 | | 100.00 | 100.00 |
| | oil | 3 | days | 99.10 | 99.33 |
| | | 7 | days | 102.77 | 102.93 |
| | | 14 | days | 96.56 | 88.79 |
| | Vitamin E | 0 | | 100.00 | 100.00 |
| | | 3 | days | 128.56 | 160.79 |
| | | 7 | days | 0.00 | 0.00 |
| | | 14 | days | 102.29 | 65.02 |
| | Miglyol | 0 | | 100.00 | 100.00 |
| | 812 | 3 | days | 98.23 | 97.01 |
| | | 7 | days | 99.31 | 96.78 |
| | | 14 | days | 99.17 | 99.48 |
| | Miglyol | 0 | | 100.00 | 100.00 |
| | 812, | 3 | days | 98.41 | 97.83 |
| | 0.02% | 7 | days | 97.43 | 98.17 |
| | BHA/BHT | 14 | days | 98.72 | 102.15 |
| | Captex | 0 | | 100.00 | 100.00 |
| | 200 | 3 | days | 99.20 | 97.28 |
| | | 7 | days | 100.14 | 97.68 |
| | | 14 | days | 108.83 | 101.15 |
| | Labrafac | 0 | | 100.00 | 100.00 |
| | CC | 3 | days | 98.60 | 95.84 |
| | | 7 | days | 100.05 | 99.51 |
| | | 14 | days | 101.37 | 100.24 |
| Hydrophilic | PEG 300 | 0 | | 100.00 | 100.00 |
| | | 3 | days | 78.22 | 18.95 |
| | | 7 | days | 52.68 | 4.61 |
| | | 14 | days | 10.09 | 1.84 |
| | Propylene | 0 | | 100.00 | 100.00 |
| | Glycol | 3 | days | 97.56 | 99.71 |
| | | 7 | days | 101.73 | 108.47 |
| | | 14 | days | 105.83 | 138.22 |
| Surfactant | Cremophor | 0 | | 100.00 | 100.00 |
| | ELP | 3 | days | 82.61 | 66.28 |
| | | 7 | days | 62.86 | 60.90 |
| | | 14 | days | 51.90 | 59.92 |
| | Cremophor | 0 | | 100.00 | 100.00 |
| | RH 40 | 3 | days | 105.30 | 91.91 |
| | 25% in | 7 | days | 92.10 | 78.30 |
| | Miglyol | 14 | days | 96.88 | 87.95 |
| | 812 | | | | |
| | Polysor- | 0 | | 100.00 | 100.00 |
| | bate 80 | 3 | days | 87.94 | 67.43 |
| | | 7 | days | 87.29 | 71.71 |
| | | 14 | days | 60.52 | 66.08 |
| | GELUCIRE | 0 | | 100.00 | 100.00 |
| | 44/14 | 3 | days | 98.70 | 107.68 |
| | 25% | 7 | days | 101.55 | 83.06 |
| | in Miglyol | 14 | days | 100.96 | 98.11 |
| | 812 | | | | |
| | Vitamin E | 0 | | 100.00 | 100.00 |
| | TPGS | 3 | days | 101.15 | 97.26 |
| | 25% | 7 | days | 101.26 | 98.74 |
| | in Miglyol | 14 | days | 103.61 | 100.15 |
| | 812 | | | | |
| | Labrifil M | 0 | | 100.00 | 100.00 |
| | | 3 | days | 98.46 | 95.19 |
| | | 7 | days | 99.45 | 95.64 |
| | | 14 | days | 100.30 | 78.97 |
| | Poloxamer | 0 | | 100.00 | 100.00 |
| | 188 | 3 | days | 116.42 | 76.47 |
| | 25% | 7 | days | 126.39 | 116.67 |
| | in Miglyol | 14 | days | 126.79 | 83.30 |
| | 812 | | | | |

The recovery data suggest that the most compatible components are Miglyol 812 (with or without BHT and BHA), Labrafac CC and Captex 200 in the lipophilic component group, propylene glycol in the hydrophilic group, and vitamin E TPGS and GELUCIRE 44/14 in the surfactant group.

### EXAMPLE 2

### Stability of Liquid and Semi-Sold Calcitriol Formulations

I. Introduction

In this Example, the stability of the active vitamin D compound calcitriol was measured in nine different formulations (four liquid formulations and five semisolid formulations).

II. Preparation of Calcitriol Formulations

A. Liquid Formulations

Four liquid calcitriol formulations (L1-L4) were prepared containing the ingredients listed in Table 2. The final formulation contains 0.208 mg calcitriol per gram of liquid formulation.

TABLE 2
Composition of Liquid Calcitriol Formulations

| Ingredient | L1 | L2 | L3 | L4 |
|---|---|---|---|---|
| Calcitriol | 0.0208 | 0.0208 | 0.0208 | 0.0208 |
| Miglyol 812 | 56.0 | 62.0 | 0 | 0 |
| Captex 200 | 0 | 0 | 55.0 | 0 |
| Labrafac CC | 0 | 0 | 0 | 55.0 |
| Vitamin-E TPGS | 15.0 | 24.0 | 22.0 | 20.0 |
| Labrifil M | 23.0 | 4.0 | 14.0 | 15.0 |
| 1,2-propylene glycol | 6.0 | 10.0 | 9.0 | 10.0 |
| BHT | 0.05 | 0.05 | 0.05 | 0.05 |
| BHA | 0.05 | 0.05 | 0.05 | 0.05 |

Amounts shown are in grams.

B. Semi-Solid Formulations

Five semi-solid calcitriol formulations (SS1-SS5) were prepared containing the ingredients listed in Table 3. The final formulation contains 0.208 mg calcitriol per gram of semi-solid formulation.

TABLE 3
Composition of Semi-Solid Calcitriol Formulations

| Ingredient | SS1 | SS2 | SS3 | SS4 | SS5 |
|---|---|---|---|---|---|
| Calcitriol | 0.0208 | 0.0208 | 0.0208 | 0.0208 | 0.0208 |
| Miglyol 812 | 80.0 | 0 | 65.0 | 0 | 79.0 |
| Captex 200 | 0 | 82.0 | 0 | 60.0 | 0 |
| Labrafac CC | 0 | 0 | 0 | 0 | 12.0 |
| Vitamin-E TPGS | 20.0 | 18.0 | 5.0 | 5.0 | 9.0 |
| Labrifil M | 0 | 0 | 0 | 0 | 0 |
| Gelucire 44/14 | 0 | 0 | 30.0 | 35.0 | 0 |
| BHT | 0.05 | 0.05 | 0.05 | 0.05 | 0.05 |
| BHA | 0.05 | 0.05 | 0.05 | 0.05 | 0.05 |

Amounts shown are in grams.

C. Method of Making the Liquid and Semi-Solid Calcitriol Formulations

1. Preparation of Vehicles

One hundred gram quantities of the four liquid calcitriol formulations (L1-L4) and the five semi-solid calcitriol formulations (SS1-SS5) listed in Tables 2 and 3, respectively, were prepared as follows.

The listed ingredients, except for calcitriol, were combined in a suitable glass container and mixed until homogeneous. Vitamin E TPGS and GELUCIRE 44/14 were heated and homogenized at 60° C. prior to weighing and adding into the formulation.

2. Preparation of Active Formulations

The semi-solid vehicles were heated and homogenized at # 60° C. Under subdued light, 12±1 mg of calcitriol was weighed out into separate glass bottles with screw caps, one bottle for each formulation. (Calcitriol is light-sensitive; subdued light/red light should be used when working with calcitriol/calcitriol formulations.) The exact weight was recorded to 0.1 mg. The caps were then placed on the bottles as soon as the calcitriol had been placed into the bottles. Next, the amount of each vehicle required to bring the concentration to 0.208 mg/g was calculated using the following formula:
C_{w}/0.208=required weight of vehicle
Where C_{w}=weight of calcitriol, in mg, and
0.208=final concentration of calcitriol (mg/g).

Finally, the appropriate amount of each vehicle was added to the respective bottle containing the calcitriol. The formulations were heated (# 60° C.) while being mixed to dissolve the calcitriol.

III. Stability of Calcitriol Formulations

The nine calcitriol formulations (L1-L4 and SS1-SS5) were analyzed for stability of the calcitriol component at three different temperatures. Sample of the nine formulations were each placed at 25° C., 40° C., and 60° C. Samples from all three temperatures for all nine formulations were analyzed by HPLC after 1, 2 and 3 weeks. In addition, samples from the 60° C. experiment were analyzed by HPLC after 9 weeks. The percent of the initial calcitriol concentration remaining at each time point was determined for each sample and is reported in Table 4 (liquid formulations) and Table 5 (semi-solid formulations).

TABLE 4
Stability of Liquid Formulations

| | | Recovery* of Calcitriol (%) | | | |
|---|---|---|---|---|---|
| Formulation | Temp. | Week 1 | Week 2 | Week 3 | Week 9 |
| Liquid #1 | 25° C. | 99.3 | 98.6 | 99.7 | ND |
| | 40° C. | 103.2 | 100.4 | 100.2 | ND |
| | 60° C. | 99.4 | 98.4 | 98.4 | 91.7 |
| Liquid #2 | 25° C. | 98.1 | 95.2 | 97.7 | ND |
| | 40° C. | 98.0 | 97.1 | 99.2 | ND |
| | 60° C. | 97.1 | 95.6 | 96.7 | 93.1 |
| Liquid #3 | 25° C. | 99.7 | 99.2 | 102.3 | ND |
| | 40° C. | 100.1 | 99.9 | 100.7 | ND |
| | 60° C. | 98.3 | 98.7 | 98.4 | 90.5 |
| Liquid #4 | 25° C. | 98.4 | 97.7 | 98.0 | ND |
| | 40° C. | 100.0 | 101.0 | 100.8 | ND |
| | 60° C. | 98.5 | 97.5 | 99.0 | 86.1 |

*Percent of time zero concentration.

TABLE 5
Stability of Semi-Solid Formulations

| | | Recovery* of Calcitriol (%) | | | |
|---|---|---|---|---|---|
| Formulation | Temp. | Week 1 | Week 2 | Week 3 | Week 9 |
| Semi-Solid #1 | 25° C. | 98.5 | 98.9 | 99.8 | ND |
| | 40° C. | 99.6 | 99.0 | 98.2 | ND |
| | 60° C. | 97.9 | 97.2 | 96.3 | 104.6 |
| Semi-Solid #2 | 25° C. | 100.0 | 99.6 | 100.4 | ND |
| | 40° C. | 98.7 | 99.6 | 98.7 | ND |
| | 60° C. | 97.2 | 98.0 | 98.6 | 100.0 |
| Semi-Solid #3 | 25° C. | 101.2 | 98.9 | 100.4 | ND |
| | 40° C. | 100.0 | 98.7 | 98.8 | ND |
| | 60° C. | 98.3 | 97.6 | 98.4 | 97.1 |
| Semi-Solid #4 | 25° C. | 100.2 | 99.0 | 99.6 | ND |
| | 40° C. | 98.4 | 99.2 | 98.5 | ND |
| | 60° C. | 96.8 | 97.7 | 97.7 | 103.4 |
| Semi-Solid #5 | 25° C. | 98.8 | 99.2 | 98.9 | ND |
| | 40° C. | 99.0 | 97.1 | 96.8 | ND |
| | 60° C. | 96.8 | 96.7 | 96.0 | 97.7 |

*Percent of time zero concentration.

As illustrated by Tables 4 and 5, calcitriol remained relatively stable with very little degradation in all of the formulations (liquid and semi-solid) analyzed.

### EXAMPLE 3

### Appearance and UV/Visible Absorption Study of Calcitriol Formulations

Calcitriol formulations L1 and SS3 were prepared prior to this study and stored at room temperature protected from light. Table 6 below shows the quantities of ingredients used to prepare the formulations.

TABLE 6
Composition of Calcitriol Formulations
Used for Absorption Analysis

| Ingredient | Liquid #1 | Semi-Solid #3 |
|---|---|---|
| Calcitriol | 0.0131 | 0.0136 |
| Vitamin-E TPGS | 9.45 | 3.27 |
| Miglyol 812 | 35.28 | 42.51 |
| Labrifil M | 14.49 | 0 |
| Gelucire 44/14 | 0 | 19.62 |
| 1,2-propylene glycol | 3.78 | 0 |
| BHA | 0.03 | 0.03 |
| BHT | 0.03 | 0.03 |

Amounts shown are in grams.

The formulations were warmed to 55° C. prior to use. Both formulations (liquid #1 and semi-solid #3) were mixed well with a vortex mixer and appeared as clear liquids. Each calcitriol formulation (0.250 μL) was added to a 25 mL volumetric flask. The exact weights added were 249.8 mg for Liquid-1 and 252.6 mg for semi-solid #3. Upon contact with the glass, the semi-solid-3 formulation became solidified. Deionized water was then added to the 25 mL mark and the solutions were mixed with a vortex mixer until uniform. The appearance was observed at this point and the absorbance of the resulting mixtures at 400 nm was determined by UV/visible spectrophotometry. Deionized water was used as a blank and the measurements were taken at 400 nm. Each sample was measured 10 times over a period of 10 minutes. The results are summarized in Table 7. Both formulations formed were white and opaque.

TABLE 7
Absorption Readings of the Formulations at 400 nm

| Measurement | Liquid #1 | Semi-Solid #3 |
|---|---|---|
| 1 | 2.4831 | 1.6253 |
| 2 | 2.5258 | 1.6290 |
| 3 | 2.5411 | 1.6309 |
| 4 | 2.5569 | 1.6328 |
| 5 | 2.5411 | 1.6328 |
| 6 | 2.5258 | 1.6347 |
| 7 | 2.5569 | 1.6328 |
| 8 | 2.5111 | 1.6366 |
| 9 | 2.5111 | 1.6366 |
| 10 | 2.5411 | 1.6328 |
| Average | 2.5294 | 1.6324 |
| RSD % | 0.91 | 0.21 |

### EXAMPLE 4

### Diameter of Emulsion Droplets Formed from the Liquid and Semi-Solid Formulation Vehicles (Without Calcitriol)

In this example, the average diameter of emulsion droplets was measured after of the liquid (L1-L4) and semi-solid (SS1-SS5) emulsion pre-concentrate vehicles (not containing calcitriol) with simulated gastric fluid (SGF) lacking enzyme. The average diameter of the droplets was determined based on light scattering measurements. The appearance of the pre-concentrates and the resulting emulsions, determined by visual inspection, was also noted. The results are summarized in Table 8.

TABLE 8
Diameter of Emulsion Droplets Formed From Emulsion Pre-
Concentrate Vehicles (without calcitriol)

| | Appearance | | | |
|---|---|---|---|---|
| | of emulsion | pre- | Ave. hydro- | |
| | pre- | concentrate: | dynamic | Appearance |
| Formulation | concentrate | SGF ratio | diameter* | of emulsion |
| L1 | Clear | 1:1600 | 237 | opaque |
| | liquid | | | |
| L2 | Clear | 1:1600 | 281 | opaque |
| | liquid | | | |
| L3 | Clear | 1:1600 | 175 | opaque |
| | liquid | | | |
| L4 | Clear | 1:1600 | 273 | opaque |
| | liquid | | | |
| SS1 | Semi-solid | 1:2000 | 305 | opaque |
| SS2 | Semi-solid | 1:2000 | 259 | opaque |
| SS3 | Semi-solid | 1:2000 | 243 | opaque |
| SS4 | Semi-solid | 1:2000 | 253 | opaque |
| SS5 | Semi-solid | 1:2000 | 267 | opaque |

*(Zaverage in nanometer)

From the results presented above, it is concluded that the droplets (particles) formed from the emulsion preconcentrate formulations were of sub-micron droplet size despite having an opaque appearance.

### EXAMPLE 5

### Diameter of Emulsion Droplets Formed from Liquid and Semi-Solid Calcitriol Formulation

In this example, the average diameter of emulsion droplets was measured after dilution of the liquid #1 (L1) and semi-solid #3 (SS3) emulsion pre-concentrates in simulated gastric fluid (SGF) without enzyme. The formulations used in this example contained calcitriol at a concentration of 0.2 mg calcitriol/g of formulation. The diameter of the droplets was determined based on light scattering measurements. The appearance of the resulting emulsions, determined by visual inspection, was also noted. The results are summarized in Table 9.

TABLE 9
Diameter of Emulsion Droplets Formed From Emulsion
Pre-Concentrate Formulations Containing Calcitriol

| | pre- | Ave. hydro- | |
|---|---|---|---|
| | concentrate: | dynamic | Appearance of |
| Formulation | SGF ratio | diameter* | emulsion |
| L1 | 1:1600 | 257 | opaque |
| SS3 | 1:2000 | 263 | opaque |

*(Zaverage in nanometer)

### EXAMPLE 6

### In Vitro Dispersion of Calcitriol from Emulsion Pre-Concentrates

In this Example, the extent of calcitriol dispersion in various formulations in gelatin capsules was determined. A single capsule containing 250 mg of a calcitriol formulation in a size-2 gelatin capsule (each capsule containing 0.2 mg calcitriol/g formulation) was added to 200 mL of simulated gastric fluid (SGF) without enzyme at 37° C. and was mixed by a paddle at 200 RPM. Samples were then filtered through a 5 μm filter and analyzed for calcitriol concentration at 30, 60, 90, and 120 minutes by HPLC. The results are shown in Table 10.

TABLE 10
Percent Calcitriol Obtained in Filtrate After Dispersion in SGF
and Filtration Through a 5 μm Filter

| Formulation | 30 min. | 60 min. | 90 min. | 120 min. |
|---|---|---|---|---|
| Liquid #1 | 106 | 103 | 86 | 68 |
| Semi-Solid #3 | 109 | 99 | 73 | 53 |
| Comparison Formulation^{#} | 0 | 0 | 0 | 0 |

^{#}The Comparison Formulation contained calcitriol at 0.2 mg/g dissolved in
Miglyol 812 with 0.05% BHA and 0.05% BHT. This formulation is similar to
the ROCALTROL formulation available from Roche Laboratories.

As this Example illustrates, the dispersion of calcitriol in simulated gastric fluid from capsules containing either the L1 or the SS3 formulations was much more extensive than that which was observed with capsules containing the Comparison Formulation (which is similar to the ROCALTROL formulation available from Roche Laboratories).

### EXAMPLE 7

### Plasma Concentrations and Pharmacokinetics of Calcitriol in Dogs

A pharmacokinetics study in dogs compared the plasma levels of calcitriol after administration of 1.0 μg/kg using 3 different formulations: ROCALTROL, a liquid formulation (liquid #1, and a semi-solid formulation (semi-solid #3). Four dogs received 1.0 μg/kg orally of ROCALTROL, the semi-solid formulation, or the liquid formulation. When dogs were used for more than one formulation a minimum 7-day washout period separated dosing with each formulation.

Blood samples were obtained pre-dose, and 0.5, 1, 2, 4, 6, 8, 10, 12, 24, 36, and 48 hours post-dose for analysis of calcitriol levels. Blood samples for clinical chemistry were obtained pre-dose, and at 24 and 48 hours post-dose for the ROCALTROL group; samples were obtained pre-dose, and at 4, 24, 48, 72, 96, and 120 hours for the semi-solid and liquid formulations. Samples were analyzed for calcitriol by radioimmunoassay and subjected to pharmacokinetics analyses.

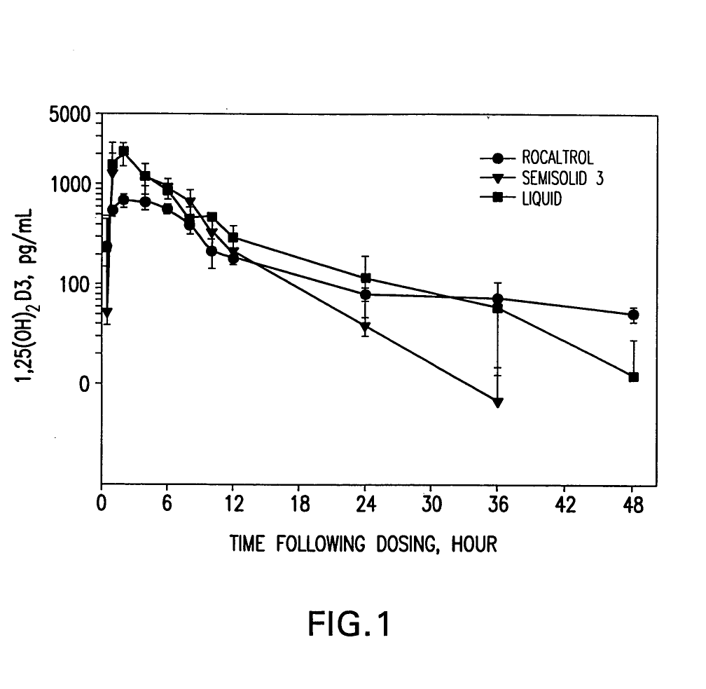

Plasma concentrations of calcitriol over time for the three formulations are shown graphically in FIG. 1.

A summary of the pharmacokinetics of calcitriol as one of three different formulations at a common dose of 1.0 μg/kg is presented in Tables 11-14.

TABLE 11
Summary of Calcitriol Parameters in Dogs

| | ROCALTROL | | Semi-Solid #3 | | Liquid #1 | |
|---|---|---|---|---|---|---|
| Parameter | Mean | SD | Mean | SD | Mean | SD |
| Cₘₐₓ, pg/mL | 717.4 | 51.5 | 2066.6 | 552.5 | 2164.4 | 253.9 |
| Tₘₐₓ^{a}, h | 3.0 | (2-6) | 2.0 | (1-2) | 1.5 | (1-2) |
| AUC₍₀₋₄₎, | 11988.0 | 3804.7 | 12351.7 | 1624.9 | 14997.4 | 3531.7 |
| pg · h/mL | | | | | | |
| T_{1/2}^{b}, h | 25.1 | 11.1 | 4.8 | 1.2 | 7.8 | 3.5 |

^{a}Expressed as median and range
^{b}Expressed as harmonic mean and pseudo SD based on jackknife variance

TABLE 12
Plasma Concentration (pg/mL) and Pharmacokinetic Parameters of Calcitriol
in Dog Following a Single 1 μg/kg Administration of ROCALTROL

| Parameter | Time, h | Dog 101 | Dog 102 | Dog 103 | Dog 104 | Mean | SD |
|---|---|---|---|---|---|---|---|
| | 0.0 | BQL | BQL | BQL | BQL | 0 | 0 |
| | 0.5 | 488.2 | 304.8 | 182.7 | BQL | 243.9 | 205.4 |
| | 1.0 | 478.2 | 634.8 | 500.7 | 555.7 | 542.4 | 69.7 |
| | 2.0 | 518.2 | 700.8 | 749.7 | 765.7 | 683.6 | 113.7 |
| | 4.0 | 494.2 | 658.8 | 750.7 | 745.7 | 662.4 | 119.8 |
| | 6.0 | 652.2 | 566.8 | 496.7 | 523.7 | 559.9 | 68.0 |
| | 8.0 | 381.2 | 366.8 | 418.7 | 381.7 | 387.1 | 22.2 |
| | 10.0 | 313.2 | 212.8 | 165.7 | 158.7 | 212.6 | 71.2 |
| | 12.0 | 190.2 | 186.8 | 189.7 | 171.7 | 184.6 | 8.7 |
| | 24.0 | 78.2 | 78.8 | 69.7 | 97.7 | 81.1 | 11.8 |
| | 36.0 | 63.2 | 83.8 | 80.7 | 67.7 | 73.9 | 10.0 |
| | 48.0 | 66.2 | 47.8 | 45.7 | 52.7 | 53.1 | 9.2 |
| Cₘₐₓ, pg/mL | | 652.2 | 700.8 | 750.7 | 765.7 | 717.4 | 51.5 |
| Tₘₐₓ^{a}, h | | 6.0 | 2.0 | 4.0 | 2.0 | 3.0 | (2-6) |
| AUC₍₀₋₄₎, | | 17693.6 | 10094.5 | 9976.2 | 10187.5 | 11988.0 | 3804.7 |
| pg · h/mL | | | | | | | |
| T_{1/2}^{b}, h | | 100.4 | 18.8 | 20.2 | 21.3 | 25.1 | 11.1 |

^{a}Expressed as median and range
^{b}Expressed as harmonic mean and pseudo SD based on jackknife variance
Bold type - used to calculate λ

TABLE 13
Plasma Concentration (pg/mL) and Pharmacokinetic Parameters of Calcitriol in Dog
Following a Single 1 μg/kg Administration of Semi-solid #3 Formulation

| Parameter | Time, h | Dog 101 | Dog 102 | Dog 103 | Dog 104 | Mean | SD |
|---|---|---|---|---|---|---|---|
| | 0.0 | BQL | BQL | BQL | BQL | 0 | 0 |
| | 0.5 | 198.1 | 11.0 | BQL | BQL | 52.3 | 97.4 |
| | 1.0 | 1208.1 | 2246.0 | 1128.7 | 503.4 | 1271.6 | 722.0 |
| | 2.0 | 1255.1 | 2110.0 | 2269.7 | 2495.4 | 2032.6 | 541.9 |
| | 4.0 | 902.1 | 1371.0 | 1095.7 | 1437.4 | 1201.6 | 248.5 |
| | 6.0 | 603.1 | 1039.0 | 932.7 | 1112.4 | 921.8 | 224.9 |
| | 8.0 | 815.1 | 441.0 | 593.7 | 848.4 | 674.6 | 192.4 |
| | 10.0 | 253.1 | 489.0 | 285.7 | 305.4 | 333.3 | 106.0 |
| | 12.0 | 213.1 | 295.0 | 184.7 | 170.4 | 215.8 | 55.7 |
| | 24.0 | 50.1 | 37.0 | 40.7 | 29.4 | 39.3 | 8.6 |
| | 36.0 | 14.1 | BQL | BQL | 13.6 | 6.9 | 8.0 |
| | 48.0 | BQL | BQL | BQL | BQL | 0.0 | 0.0 |
| Cₘₐₓ, pg/mL | | 1255.1 | 2246.0 | 2269.7 | 2495.4 | 2066.6 | 552.5 |
| Tₘₐₓ^{a}, h | | 2.0 | 1.0 | 2.0 | 2.0 | 2.0 | (1-2) |
| AUC₍₀₋₄₎, pg · h/mL | | 10333.8 | 14012.9 | 11813.8 | 13246.4 | 12351.7 | 1624.9 |
| T_{1/2}^{b}, h | | 6.2 | 3.8 | 4.1 | 5.9 | 4.8 | 1.2 |

^{a}Expressed as median and range
^{b}Expressed as harmonic mean and pseudo SD based on jackknife variance
Bold type - used to calculate λ

TABLE 14
Plasma Concentration (pg/mL) and Pharmacokinetic Parameters of Calcitriol
in Dogs Following a Single 1 μg/kg Liquid #1 Formulation

| Parameter | Time, h | Dog 105 | Dog 106 | Dog 107 | Dog 108 | Mean | SD |
|---|---|---|---|---|---|---|---|
| | 0.0 | BQL | BQL | BQL | BQL | 0 | 0 |
| | 0.5 | BQL | 57.6 | 523.0 | 350.0 | 232.7 | 246.9 |
| | 1.0 | 1283.0 | 238.6 | 2266.0 | 2468.0 | 1563.9 | 1024.0 |
| | 2.0 | 2028.0 | 1895.6 | 2026.0 | 2373.0 | 2080.7 | 204.5 |
| | 4.0 | 1090.0 | 892.6 | 1009.0 | 1771.0 | 1190.7 | 395.3 |
| | 6.0 | 871.0 | 763.6 | 730.0 | 1063.0 | 856.9 | 150.0 |
| | 8.0 | 301.0 | 579.6 | 374.0 | 562.0 | 454.2 | 138.1 |
| | 10.0 | 421.0 | 520.6 | 464.0 | 517.0 | 480.7 | 47.4 |
| | 12.0 | 348.0 | 290.6 | 170.0 | 373.0 | 295.4 | 90.4 |
| | 24.0 | 42.0 | 165.6 | 62.0 | 202.0 | 117.9 | 78.0 |
| | 36.0 | 49.0 | 111.6 | BQL | 79.0 | 59.9 | 47.4 |
| | 48.0 | 35.0 | 15.5 | BQL | BQL | 12.6 | 16.6 |
| Cₘₐₓ, pg/mL | | 2028.0 | 1895.6 | 2266.0 | 2468.0 | 2164.4 | 253.9 |
| Tₘₐₓ^{a}, h | | 2.0 | 2.0 | 1.0 | 1.0 | 1.5 | (1-2) |
| AUC₍₀₋₄₎, | | 13474.4 | 14296.3 | 12101.0 | 20117.7 | 14997.4 | 3531.7 |
| pg · h/mL | | | | | | | |
| T_{1/2}^{b}, h | | 10.6 | 8.5 | 5.0 | 10.1 | 7.8 | 3.5 |

^{a}Expressed as median and range
^{b}Expressed as harmonic mean and pseudo SD based on jackknife variance
Bold type - used to calculate λ

The results of this study show that there were some differences and similarities in the pharmacokinetics between these particular inventive formulations and ROCALTROL as follows:
Cₘₐₓ was approximately three times higher with the liquid and semi-solid formulations than with the ROCALTROL formulation.
Cₘₐₓ was achieved sooner (1 to 2 hours) with the liquid and semi-solid formulations than with the ROCALTROL formulation (2 to 4 hours).
The overall systemic exposure (AUC₀₋₄) was comparable with the three formulations, although systemic exposure in the first 24-48 hours was greater with the liquid and semi-solid formulations than with ROCALTROL.

The foregoing results show that the liquid #1 formulation produces the highest Cₘₐₓ and the largest AUC calcitriol values, followed closely by the semi-solid #3 formulation. The ROCALTROL formulation has the lowest Cₘₐₓ and AUC values. It appears that the liquid #1 and semi-solid #3 formulations were absorbed much faster and produced higher plasma concentration during the first twelve hours and a faster rate of elimination.

### EXAMPLE 8

### Pharmacokinetics of the Semi-Solid #3 Formulation After Escalating Doses

In this study the pharmacokinetics of the semi-solid formulation after escalating oral doses was studied in dogs. Three male and three female Beagle dogs were dosed orally with single doses of 0.5 μg/kg (all six dogs), 0.1 μg/kg (1 male and 1 female), 5.0 μg/kg (2 males and 2 females), and 10.0 μg/kg (all dogs). After the 10.0 μg/kg dose, 2 dogs per sex were euthanized. The remaining male and female dogs continued on study and received doses of 30.0 μg/kg and 100.0 μg/kg. After each dose the animals were held for a 6-day recovery period.

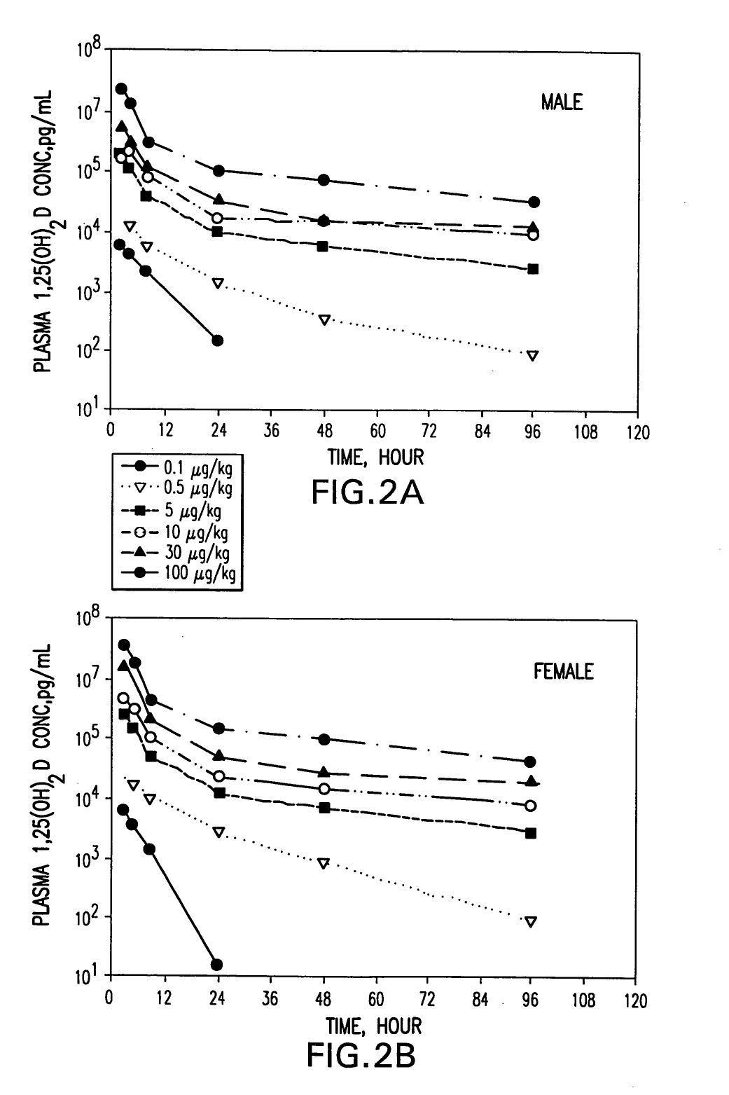

Blood samples (approximately 1 mL) were collected from each dog pre-dose and at 0, 2 (in all but the 0.5 μg/kg dose), 4, 8, 24, 48, and 96 hours following dose administration. Samples were analyzed for calcitriol by radioimmunoassay and subjected to pharmacokinetic analyses. Plasma concentrations of calcitriol are shown graphically for males and females in FIGS. 2A and 2B.

After dosing with semi-solid #3, maximum plasma concentrations usually occurred at the two hour sampling timepoint. At doses above 0.1 μg/kg, plasma concentrations appeared to decline at a more rapid rate during the first 8 hours than during the 24 to 96 hour time period.

At the lowest dose of 0.1 μg/kg, plasma concentrations of calcitriol fell below the limit of quantitation after 24 hours. At 0.5 μg/kg and above, measurable concentrations of calcitriol persisted at the 96 hour sampling timepoint. There did not appear to be any remarkable differences between the male and the female dogs.

Pharmacokinetic parameters for semi-solid #3 at doses ranging from 0.1 to 100.0 μg/kg are summarized in Table 15.

TABLE 15
Pharmacokinetics of Calcitriol After Escalating Doses of Calcitriol (Semi-solid #3)

| Dose | | |
|---|---|---|
| (μg/kg) | | |
| 0.1 | 0.5 | 5.0 |

| | | | | | | |
|---|---|---|---|---|---|---|
| Gender | Male | Female | Male | Female | Male | Female |
| N | 1 | 1 | 3 | 3 | 2 | 2 |
| Cₘₐₓ(pg/mL) | 566 | 473 | 1257 | 1431 | 17753 | 18346 |
| Tmax (hr) | 2.0 | 2.0 | 4.0 | 4.0 | 2.0 | 2.0 |
| AUC₀₋₂₄ | 4311 | 2654 | 11431 | 15598 | 104,027 | 107,452 |
| (pg · hr/mL) | | | | | | |
| AUC₀₋₄₈ | 4311 | 2654 | 13584 | 19330 | 125,408 | 126,746 |
| (pg · hr/mL) | | | | | | |
| AUC₀₋₄ | 4916 | 2718 | 15062 | 21644 | 200,283 | 160,681 |
| (pg · hr/mL) | | | | | | |
| T_{1/2}(hr) | 4.2 | 2.7 | 17.1 | 14.2 | 67.6 | 36.8 |

| | Dose | | | | | |
|---|---|---|---|---|---|---|
| | (μg/kg) | | | | | |
| | 10.0 | | 30.0 | | 100.0 | |
| Gender | Male | Female | Male | Female | Male | Female |
| N | 3 | 3 | 1 | 1 | 1 | 1 |
| Cₘₐₓ | 23858 | 32336 | 53005 | 115,896 | 238,619 | 211,631 |
| (pg/mL) | | | | | | |
| Tmax (hr) | 2.7 | 2.0 | 2.0 | 2.0 | 2.0 | 2.0 |
| AUC₀₋₂₄ | 183,981 | 203,857 | 311,841 | 567,717 | 1,165,988 | 1,089,831 |
| (pg · hr/mL) | | | | | | |
| AUC₀₋₄₈ | 223,977 | 240,483 | 370,713 | 641,469 | 1,381,424 | 1,256,007 |
| (pg · hr/mL) | | | | | | |
| AUC₀₋₄ | 388,600 | 345,936 | 531,303 | 854,841 | 1,874,997 | 1,731,873 |
| (pg · hr/mL) | | | | | | |
| T_{1/2}(hr) | 77.7 | 56.0 | 56.3 | 58.2 | 45.3 | 53.7 |

These pharmacokinetic results indicate the following:
The systemic exposure of calcitriol appeared to be fairly linear throughout the tested dose range of 0.1 to 100.0 μg/kg. No saturation of absorption was observed.
The half-life of calcitriol appeared to be dose-dependent. Formulations having a half life of greater than 24 hours are less suitable for high dose pulse administration.
Weekly dosing with semi-solid #3 at 5.0 μg/kg and above resulted in some accumulation in the plasma. Accumulation was not consistently observed at the lower doses of 0.1 and 0.5 μg/kg.

### EXAMPLE 9

### A 28 Day Oral Toxicity Study in Dogs with Semi-Solid #3

In this study a 28-day repeated dose toxicology study of semi-solid #3 was conducted in dogs to assess the pharmacokinetics of calcitriol after weekly oral capsule dosing. Semi-solid #3 or control article capsules were administered on study days 0, 7, 14, 21, and 28. Twelve dogs (6 male, 6 female) received vehicle control (group 1), eight dogs (4 male, 4 female) received 0.1 μg/kg semi-solid #3 (group 2), and eight dogs (4 male, 4 female) received 1.0 μg/kg semi-solid #3 (group 3). Twelve dogs (6 male, 6 female) received 30.0 μg/kg semi-solid #3 on day 0 (group 4). Due to the severity of the clinical response observed after the first 30 μg/kg dose on day 0, dose levels were reduced in this group to 10 μg/kg (males on days 7, 14, 21, and 28) or 5 μg/kg (females on days 7, 14, 21, and 28). Blood samples were collected on each dog pre-dose and at 1, 2, 4, 6, 8, 24, and 48 hours following dosing on study days 0 (first dose) and 21 (fourth weekly dose). All animals were sacrificed on study day 29.

The pharmacokinetic results for plasma calcitriol for groups 2-4 are summarized in Table 16.

TABLE 16
Mean Toxicokinetic Parameters of Calcitriol After
Weekly Dosing with Semi-Solid #3 in Dogs
DAY 0

| | 0.1 μg/kg | | 1.0 μg/kg | | 30.0 μg/kg | |
|---|---|---|---|---|---|---|
| Dose | (Group 2) | | (Group 3) | | (Group 4) | |
| Sex (No. of | Male (4) | Female (4) | Male (4) | Female (4) | Male (6) | Female (6) |
| Dogs) | | | | | | |
| C_{max,} pg/mL | 198.7 | 430.8 | 2385.0 | 3419.1 | 84909.1 | 57133.3 |
| Tₘₐₓ^{a}, h | 1.0 | 2.0 | 1.0 | 1.5 | 2.0 | 2.0 |
| AUC₀₋₂₄, | 1840.6 | 3093.4 | 17144.2 | 23259.7 | 496044.6 | 323573.1 |
| pg · hr/mL | | | | | | |
| AUC₀₋₄₈, | 2130.8 | 3093.4 | 19141.6 | 25794.5 | 644064.2 | 365340.7 |
| pg · hr/mL | | | | | | |

DAY 24 (Fourth Weekly Dose)

| | 0.1 μg/kg | | 1.0 μg/kg | | 10.0 μg/kg | 5.0 μg/kg |
|---|---|---|---|---|---|---|
| Dose | (Group 2) | | (Group 3) | | (Group 4) | (Group 4) |
| Sex (No. | Male (4) | Female (4) | Male (4) | Female (4) | Male (6) | Female (6) |
| of Dogs) | | | | | | |
| Dose | 0.1 | 0.1 | 1.0 | 1.0 | 10.0^{b} | 5.0^{b} |
| Cₘₐₓ, pg/mL | 217.6 | 398.3 | 2272.1 | 2188.6 | 29061.8 | 8670.7 |
| Tₘₐₓ^{a}, h | 1.0 | 2.0 | 1.5 | 2.0 | 1.0 | 2.0 |
| AUC₀₋₂₄, | 1956.2 | 3283.0 | 19765.4 | 12947.3 | 173597.2 | 46878.1 |
| pg · hr/mL | | | | | | |
| AUC₀₋₄₈, | 2225.9 | 3640.7 | 24606.9 | 15380.0 | 209732.1 | 54976.1 |
| pg · hr/mL | | | | | | |

^{a}The values for Tₘₐₓ are the median values for this parameter. All other parameters
shown are mean values.
^{b}Doses of semi-solid #3 were lowered beginning on Study Day 7.
Data from the vehicle control dogs (Group 1) were not subjected to pharmacokinetic
analysis.

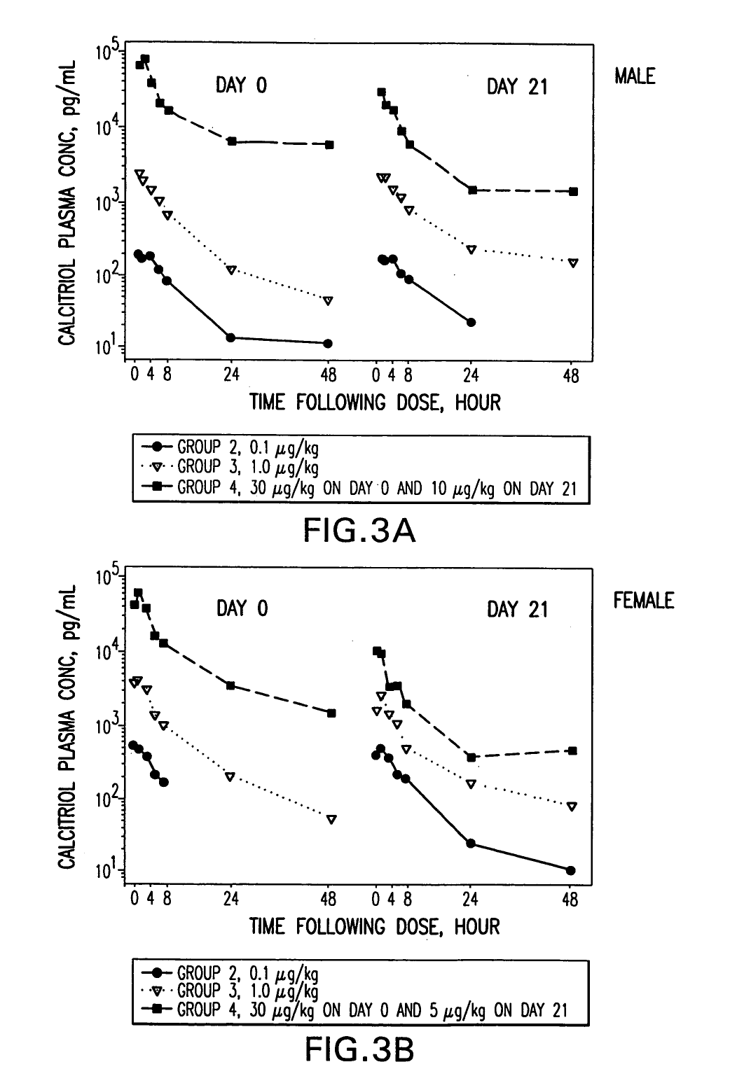

FIGS. 3A and 3B show the adjusted plasma concentration-time curve for calcitriol after oral capsule dosing with semi-solid #3 on study days 0 and 21 in male (FIG. 3A) and female (FIG. 3B) Beagle dogs. Calcitriol values at time 0 on day 0 were subtracted from all subsequent timepoints to adjust for endogenous (baseline) plasma calcitriol.

The results of the study indicate that following:
After oral capsule dosing with semi-solid #3, plasma concentrations of calcitriol rose fairly rapidly, reaching peak plasma concentrations within two hours.
Plasma concentrations of calcitriol decreased at a more rapid rate during the first 8 hours post-dosing than during the later timepoints (24-48 hours), possibly indicating redistribution of calcitriol to extravascular spaces, with subsequent slow release of calcitriol back into the vascular spaces. This observation was more apparent at the higher dose levels than at the lower dose levels.
At 24 hours post-dosing, plasma concentration of calcitriol had declined to near-baseline values at the low dose of 0.1 μg/kg. However, at the higher doses of calcitriol, dose-related residual concentrations of calcitriol were still evident at the last sampling timepoint (48 hours), although all values returned to pre-dose (baseline) values by one week post-dosing.
Values for Cₘₐₓ and AUC were fairly proportional to dose throughout the dose range tested (0.1-30.0 μg/kg).
Values for AUC₀₋₂₄ at the low dose, which was the no observable adverse effect level (0.1 μg/kg) ranged from 1840.6-3283.0 pg·hr/mL.
Values for AUC₀₋₂₄ at the mid dose, which was the maximum tolerated dose (1.0 μg/kg) ranged from 12,947.3-23,259.7 pg·hr/mL.
Values for AUC₀₋₂₄ at doses associated with weight loss and moderate signs of toxicity, ranged from 46,878.1 pg·hr/mL (5.0 μg/kg; females) to 173,597.2 pg·hr/mL (10.0 μg/kg; males).
Values for AUC₀₋₂₄ at a dose associated with mortality (30.0 μg/kg) ranged from 323,573.1-496,044.6 pg·hr/mL.
There were no consistent sex differences in any pharmacokinetic parameter.

Overall, the animals appeared to handle calcitriol similarly after the first dose and after repeated once-weekly dosing, with a few exceptions such as higher values for Cₘₐₓ and AUC on Day 0 compared to Day 21 in the 1.0 μg/kg females (not evident in the males).

### EXAMPLE 10

### Acute Toxicity Study of Three Different Formulations

In the study described in Example 7, several in-life parameters, including clinical chemistry parameters, were monitored to assess the toxicity of the calcitriol formulations. Blood samples were analyzed for calcium, phosphorus, blood urea nitrogen (BUN), glucose, albumin, bilirubin (total), aspartate aminotransferase (AST), alanine aminotransferase (ALT), alkaline phosphatase (AP), and creatinine.

No clinical toxicity was seen in any dog with any of the three formulations.

Hypercalcemia was seen after dosing with 1.0 μg/kg with all three formulations. The group mean and the individual range of serum calcium levels of each of the three different formulations are presented in Table 17.

TABLE 17
Group Mean Serum Calcium Levels (mg/dL)

| Historical | | | | | | | | |
|---|---|---|---|---|---|---|---|---|
| Control | | 0 hr | 4 hr | 24 hr | 48 hr | 72 hr | 96 hr | 120 hr |
| | | ROCALTROL, 1.0 μg/kg | | | | | | |
| 9.25-11.3^{a} | Mean | 11.1 | NA | 13.8* | 12.9* | NA | NA | NA |
| (10.44)^{b} | | | | | | | | |
| | SD | 0.31 | NA | 0.83 | 0.26 | NA | NA | NA |
| | Range | 10.8-11.5 | NA | 13.2-15.0 | 12.6-13.1 | NA | NA | NA |
| | | Calcitriol, liquid, 1.0 μg/kg | | | | | | |
| 9.25-11.3 | Mean | 10.4 | 10.5 | 16.1* | 14.3* | 12.7* | 12.5* | 12.0* |
| (10.44) | | | | | | | | |
| | SD | 0.17 | 0.37 | 1.47 | 1.34 | 0.53 | 0.78 | 0.80 |
| | Range | 10.2-10.5 | 10.1-10.9 | 13.9-17.0 | 12.9-15.7 | 12.0-13.3 | 11.5-13.4 | 11.2-13.1 |
| | | Calcitriol, semi-solid, 1.0 μg/kg | | | | | | |
| 9.25-11.3 | Mean | 10.1 | 10.6 | 14.3* | 14.2* | 12.3* | 12.6* | 12.7* |
| (10.44) | | | | | | | | |
| | SD | 0.33 | 0.29 | 1.72 | 1.52 | 1.35 | 0.76 | 0.47 |
| | Range | 9.7-10.5 | 10.7-10.8 | 12.2-16.4 | 12.1-15.5 | 10.8-13.6 | 11.5-13.1 | 12.0-13.0 |

^{a}Historical range
^{b}Historical mean
*Mean outside historical range
NA = not available (serum sample not taken)

In addition to elevations of calcium, elevations of ALT, AST, BUN, and creatinine were observed in all groups.

In summary, the results of this study indicated that:
No treatment-related clinical signs were evident in any dog after dosing with any of the formulations (ROCALTROL, liquid, or semi-solid).
Hypercalcemia at 1.0 μg/kg PO was seen in dogs with all three formulations.
Time course of the hypercalcemia was comparable among all three formulations up to 48 hours; sampling for the ROCALTROL group did not extend beyond 48 hours.
Severity of the hypercalcemia was comparable among the three formulations; the highest serum calcium (17.0 mg/dL) occurred at 24 hours in dogs receiving the liquid formulation.
Mean values for ALT, AST, BUN, and creatinine were observed to be outside the historical range in all treatment groups at one or more timepoints.
Elevations for BUN and creatinine were greater in the liquid or semi-solid groups; in the absence of a concurrent control group, the significance of this observation is unclear.

### EXAMPLE 11

### Acute Maximum Tolerated Dose Study

In the study described above in Example 8, the acute toxicity and hypercalcemia effects of semi-solid #3 were also assessed to estimate the maximum tolerated dose and to provide data for dose selection of future studies.

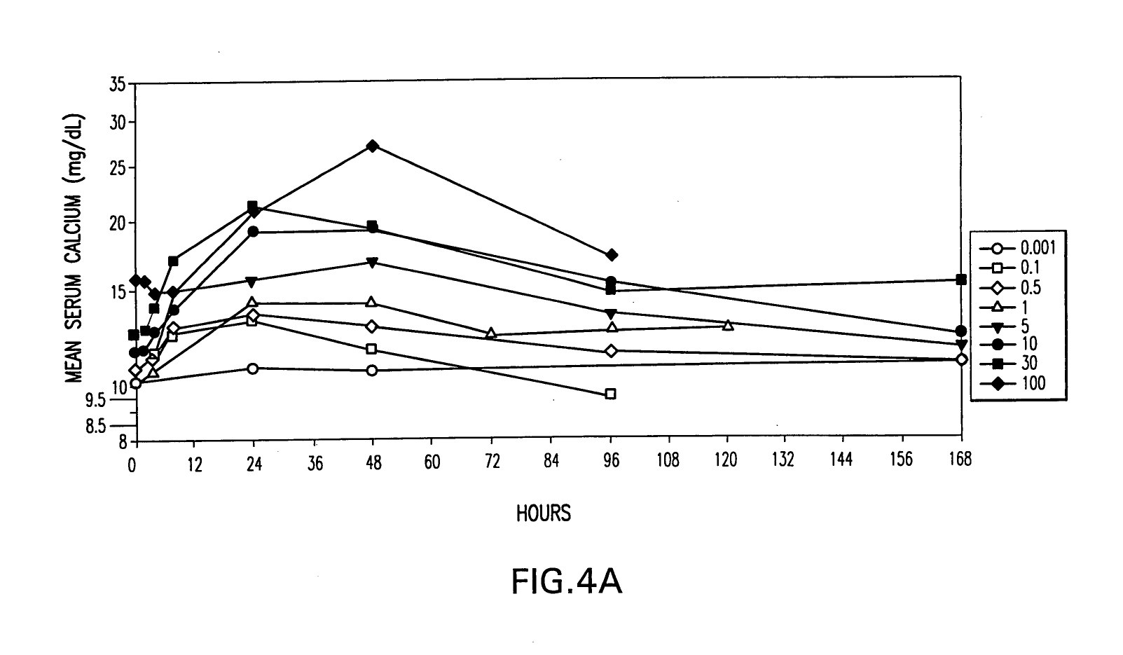

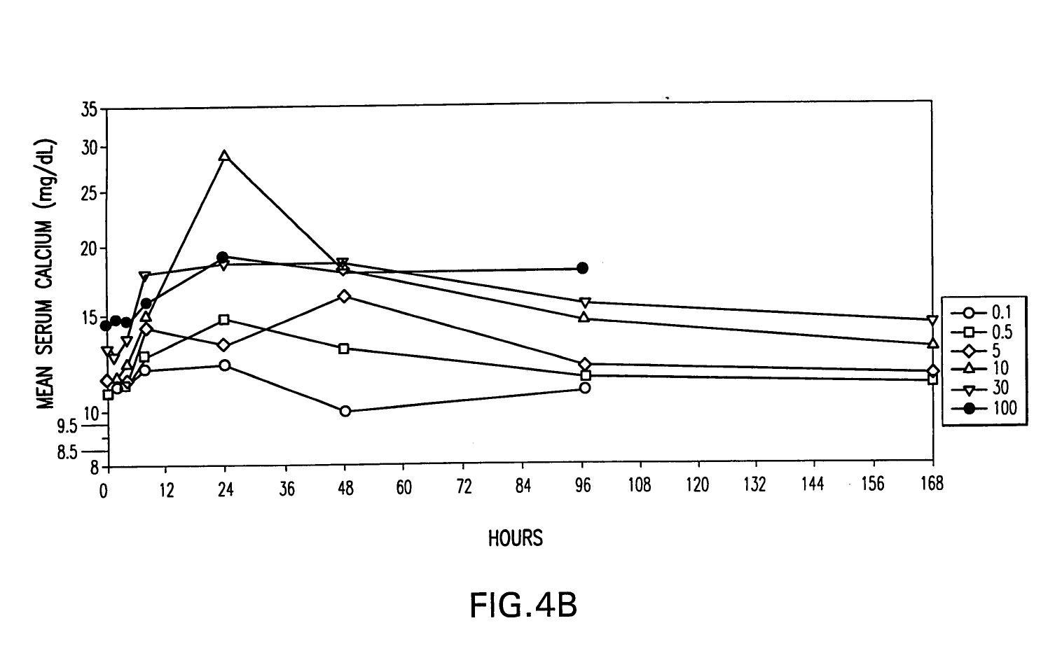

Calcium levels were increased in a dose-related manner at all dose levels in males (FIG. 4A) and females (FIG. 4B). Serum calcium data for the 0.001 and 1.0 μg/kg dose was obtained in male dogs in the study describe in Example 10, and is included here for completeness.

In summary, this study of semi-solid #3 administered orally via a capsule to male and female Beagle dogs at 0.1, 0.5, 5.0, 10.0, 30.0, and 100.0 μg/kg showed:
Dose dependent hypercalcemia was the most common laboratory abnormality.
Elevations of creatinine, urea nitrogen, cholesterol, erythrocytes, hemoglobin, hematocrit, and neutrophils, and a decrease in lymphocytes were seen at doses of 5.0 μg/kg or higher.
Body weights and food consumption decreased markedly after receiving the 30.0 and 100.0 μg/kg doses; after 100.0 μg/kg, dogs had a noticeable thin appearance and obvious decreased activity.

Based on these results, the maximum tolerated dose of semi-solid #3 in dogs appeared to be 5.0 μg/kg.

### EXAMPLE 12

### A 28 Day Repeated Dose Toxicity Study

In the study described above in Example 9, the dogs were also assessed for potential toxicity of the semi-solid #3 formulation when administered to dogs by the oral (capsule) route once every seven days for 28 days. The study included assessments of clinical signs, body weights, food consumption, toxicokinetics, clinical pathology including biochemistry, hematology, coagulation, and urinalysis, ophthalmology, cardiology, gross necropsy, organ weight, and full histopathology on all animals. The study design is summarized in Table 18.

TABLE 18
Study Design for 28-Day Repeated Dose Study in Dogs

| | No. of Main | | | | |
|---|---|---|---|---|---|
| | (Recovery) | | | | |
| | Animals | | | Bulk Dose Level | Calcitriol Dose |
| Group | Males | Females | Dose Materials | (mg/kg/dose)* | Level (μg/kg/dose) |
| 1 | 4(2) | 4(2) | Control Article | 300** | 0 |
| 2 | 4 | 4 | Test Article* | 1 | 0.1 |
| 3 | 4 | 4 | Test Article* | 10 | 1 |
| 4 | 4(2) | 4(2) | Test Article* | 300/100 | 30/10 (Males)** |
| | | | | (males) | |
| | | | | 300/50** | 30/5 (Females)** |
| | | | | (females)** | |

*The test article (calcitriol semi-solid #3) is a formulation containing 0.1 mg of calcitriol per gram.
**Dose reduced to 10 μg/kg in males and 5 μg/kg in females at Week 2; all surviving animals were sacrificed on Day 29.

Four of the group 4 animals (1 male and 3 females) died or were euthanized moribund during the first three days of the study. No deaths occurred following reduction of the dose level on day 7; there were no deaths in groups 1, 2 or 3.

In the group 4 animals that died, the most notable clinical abnormalities preceding death primarily included red vomitus, few/no feces, soft stools containing red material, red nasal discharge, shallow/rapid breathing, decreased activity and lateral recumbency.

Dose-related body weight loss, decreased weight gain, and decreased food consumption were observed in group 3 and 4 animals; group 3 animals were ˜11-12% below controls; group 4 animals were 17-24% below controls. No effects on weight gain or food consumption were apparent in group 2 animals.

There was a trend towards an increase in several RBC and WBC parameters in the group 4 animals at day 29; no toxicologically significant hematological abnormalities were apparent in the group 2 and 3 animals.

Dose related hypercalcemia was noted in group 3 and 4 animals. Calcium levels were increased by 6 hours post-dose, achieved a maximum by 24 hours post-dose, and decreased gradually at 48 and 96 hours post-dose. Other clinical chemistry abnormalities, in group 3 and 4 animals included increased serum proteins, cholesterol and kidney function parameters and decreased electrolytes and urine specific gravity. No toxicologically significant clinical chemistry abnormalities or notable increases in serum calcium were observed in group 2 animals.

There were no treatment-related changes observed in the ocular tissues on study days 22/23 and there were no treatment-related changes observed in the ECG and blood pressure data obtained on this study.

The most notable gross necropsy abnormalities occurred in group 4 animals that were found dead or were euthanized and included lesions in the digestive system and related organs; dark red omentum, reddened to dark red mucosa, red fluid in the small intestine and stomach, reddened to dark red mucosa in the esophagus and large intestine, stained and thickened gall bladder, a thrombus in the heart, dark red and mottled areas on the lungs, a reddened to dark red pancreas, a dark red thymus, thickened urinary bladder and a pale spleen. Gross abnormalities were less severe in group 3 animals; no notable gross abnormalities were observed in the group 2 animals.

The primary histopathological abnormality was dose related chronic interstitial nephritis: mild to moderate in group 3 animals and moderate to marked in group 4 animals. Other microscopic findings in these animals appeared to be secondary to chronic interstitial nephritis and included mineralization of various organs/tissues. No microscopic lesions were observed in the group 2 animals.

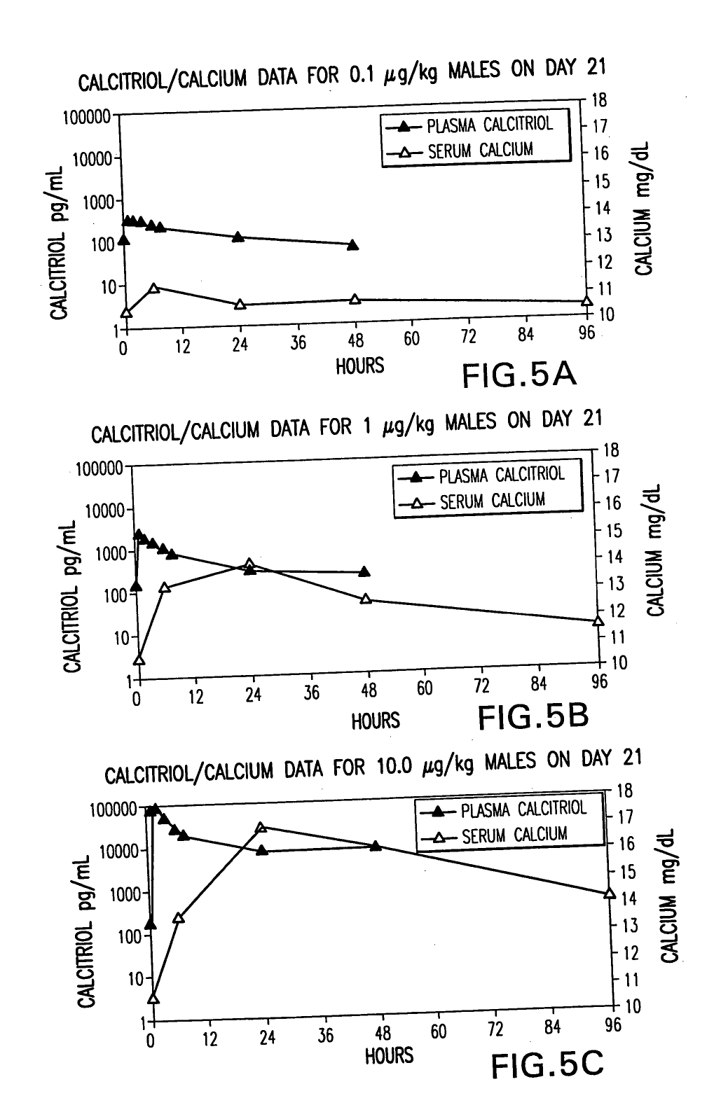

The highest values for serum calcium usually occurred within 24 hours post-dose and returned to baseline levels by the next pre-dose sampling interval. Selected data (males on Day 21) for serum calcium along with plasma calcitriol are shown in FIGS. 5A-5C. These data show that the maximum plasma concentrations of calcitriol usually occurred well in advance of the maximum serum concentrations of calcium.

In summary, this study of semi-solid #3 administered orally to dogs once every 7 days to male and female Beagle dogs at 0, 1.0 and 5.0 (females) or 10.0 (males) μg/kg following the initial dose of 30.0 μg/kg showed:
The no observed adverse effect level was 0.1 μg/kg; the maximum tolerated dose was 1.0 μg/kg; mortality was seen at 30 μg/kg.
Dose related lesions in the digestive system and related organs, reduced weight gain and decreased food consumption were seen in groups 3 and 4.
Dose related chronic interstitial nephritis was seen in groups 3 and 4.

### EXAMPLE 13

### Human Pharmacokinetic Study

Pharmacokinetics of semi-solid #3 in humans was evaluated in a clinical trial. Patients received semi-solid #3 on this study at doses of calcitriol up to 90 μg. Preliminary pharmacokinetic results are discussed below.

Blood samples were obtained pre-dose and at 0.5, 1.0, 1.5, 2, 3, 4, 6, 8, 12, 24, 48 and 72 hours post initial dose of semi-solid #3. Calcitriol levels were analyzed using a commercial radioimmunoassay, with limited validation for dilution integrity.

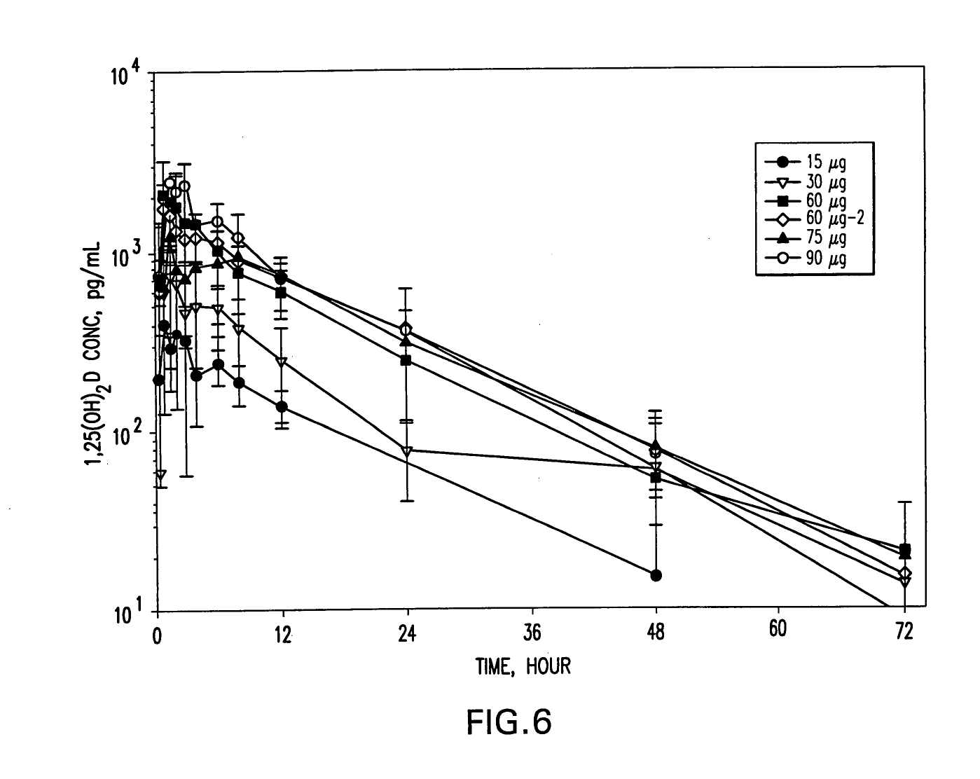

Mean plasma concentration-time curves were plotted for each group (FIG. 6). Non-compartmental pharmacokinetic parameters were calculated for each subject and then averaged (Table 19). Baseline calcitriol values were subtracted from the post-dosing values to adjust for endogenous calcitriol.

TABLE 19
Semi-Solid #3 Pharmacokinetic Parameters by Dose Group

| | | Tmax, h | | | | |
|---|---|---|---|---|---|---|
| | Cₘₐₓ, | (median | AUC_{0-24 H}, | AUC_{0-48 H}, | AUC_{0-□ H}, | |
| Dose, | pg/mL | and | pg · h/mL | pg · h/mL | pg · h/mL | |
| μg | (±SD) | range) | (±SD) | (±SD) | (±SD) | t_{1/2}, h* |
| 5.0 | 398.3 | 1.00 | 3665.7** | 5627.3*** | 5464.8 | 8.9 |
| (n = 3) | (12.9) | (1-1) | | (637.1) | (892.8) | |
| 0.0 | 898.8 | 1.50 | 6955.9 | 9792.4 | 11069.7*** | 16.3*** |
| (n = 3) | (333.6) | (1.5-2) | (2825.4 | (2323.9) | (1406.4) | |
| 0.0 | 2077.3 | 4.00 | 17480.6 | 20999.4 | 21795.0 | 7.3 |
| (n = 6) | (533.3) | (1.5-4) | (2989.7) | (4762.5) | (5124.8) | |
| 0.0 | 1918.4 | 1.3 | 17523.1 | 20663.5 | 24997.6 | 8.6 |
| (n = 4) | (605.2) | (1-1.5) | (1217.2) | (1832.1) | (4612.5) | |
| 5.0 | 1586.2 | 1.5 | 16499.1 | 21159.1 | 22690.4 | 10.8 |
| (n = 3) | (328.6) | (1-4) | (2343.8) | (3406.0) | (9209.4) | |
| 0.0 | 2858.7 | 1.5 | 23127.5 | 28164.3 | 29204.1 | 8.8 |
| (n = 3) | (496.3) | (1-2) | (5755.7) | (8428.3) | (9209.4) | |

*harmonic mean, based on jackknife variance;
**n = 1;
***n = 2

Based on these data, pharmacokinetics of semi-solid #3 appear linear and predictable. There was no evidence of saturation of absorption.

### EXAMPLE 14

### Safety Results with Semi-Solid #3

The safety of semi-solid #3 in humans was evaluated in a clinical trial. As of May 8, 2002, 12 patients received semi-solid #3 on this study: 3 in group 1 (15 μg), 3 in group 2 (30 μg), and 6 in group 3 (60 μg). Preliminary pharmacokinetic results on the first 9 patients are discussed below.

No deaths have occurred. Thirty-four (34) adverse events occurred in 8 of the 9 patients; 20 of 34 adverse events were deemed possibly of probably related to semi-solid #3. One serious adverse event occurred in group 3 that was deemed not related by the Investigator. This patient developed a transient grade 1 fever on day 1 that prolonged hospitalization. Grade 2 or 3 adverse events deemed related to study drug are presented in Table 20.

TABLE 20
Grade 2 or 3 Adverse Events Deemed Related to Study Drug

| Patient | Dose Group | Event | Severity | Comments |
|---|---|---|---|---|
| 002-1002 | 60 μg | Hyperglycemia | Grade 2 | — |
| | | Hypoproteinemia | Grade 2 | — |
| 002-1003 | 60 μg | Constipation | Grade 2 | — |
| | | Hyponatremia | Grade 3 | Sodium 127 |
| | | | | meq/L on day 4; |
| | | | | transient; no |
| | | | | intervention |

The preliminary results from the phase 1 trial with semi-solid #3 demonstrate:
The maximum tolerated dose of semi-solid #3 has not yet been determined in the phase 1 trial; additional patients are being evaluated in group 3 (60 μg).
Pharmacokinetics of semi-solid #3 appeared linear and predictable across the first three dose groups.

### EXAMPLE 15

### Additional Compositions

When semi-solid #3 was prepared in hard gelatin capsules for oral dosing, leakage of the composition from the capsules was observed. New compositions comprising different lipophilic phase components and surfactants and different percentages of each component were tested to identify compositions that would solve this problem. The compositions are listed in table 21.

TABLE 21
Additional tested compositions

| | Percent by Weight | | | | | | | | |
|---|---|---|---|---|---|---|---|---|---|
| Formulation | a | b | c | d | e | f | g | h | i |
| Gelucire | 50 | 50 | 50 | 40 | 40 | 30 | 20 | | 60 |
| 44/14 | | | | | | | | | |
| Vitamin E | | 10 | 20 | 30 | 20 | 30 | 30 | 50 | 25 |
| TPGS | | | | | | | | | |
| Miglyol 812 | 50 | 40 | 30 | 30 | 40 | 40 | 50 | 50 | 15 |
| | Percent by Weight | | | | | | | | |
| Formulation | j | k | l | m | n | o | p | q | r |
| Gelucire | 30 | 50 | 50 | 50 | 40 | 40 | 30 | 20 | 60 |
| 50/13 | | | | | | | | | |
| Vitamin E | 5 | | 10 | 20 | 30 | 20 | 30 | 30 | 25 |
| TPGS | | | | | | | | | |
| Miglyol 812 | 65 | 50 | 40 | 30 | 30 | 40 | 40 | 50 | 15 |

Percent by Weight

| Formulation | s | t | u | v | w |
|---|---|---|---|---|---|
| Gelucire | 50 | | | 33.3 | |
| 44/14 | | | | | |
| Gelucire | | 50 | | | 33.3 |
| 50/13 | | | | | |
| Vitamin E | | | 50 | 33.3 | 33.3 |
| TPGS | | | | | |
| PEG 4000 | 50 | 50 | 50 | 33.3 | 33.3 |

Additional compositions containing multiple surfactants without a lipophilic phase component were also tested. The compositions were 1:1 combinations of vitamin E TPGS with either Gelucire 44/14 or Gelucire 50/13.

Compositions that were resistant to leakage were identified.

### EXAMPLE 16

### Stable Unit Dose Formulations

Formulations of calcitriol were prepared to yield the compositions in Table 22. The Vitamin E TPGS was warmed to approximately 50° C. and mixed in the appropriate ratio with MIGLYOL 812. BHA and BHT were added to each formulation to achieve 0.35% w/w of each in the final preparations.

TABLE 22
Calcitriol formulations

| | MIGLYOL | Vitamin E TPGS |
|---|---|---|
| Formulation # | (% wt/wt) | (% wt/wt) |
| 1 | 100 | 0 |
| 2 | 95 | 5 |
| 3 | 90 | 10 |
| 4 | 50 | 50 |

After formulation preparation, Formulations 2-4 were heated to approximately 50° C. and mixed with calcitriol to produce 0.1 μg calcitriol/mg total formulation. The formulations contained calcitriol were then added (˜250 μL) to a 25 mL volumetric flask and deionized water was added to the 25 mL mark. The solutions were then vortexed and the absorbance of each formulation was measured at 400 nm immediately after mixing (initial) and up to 10 min after mixing. As shown in Table 23, all three formulations produced an opalescent solution upon mixing with water. Formulation 4 appeared to form a stable suspension with no observable change in absorbance at 400 nm after 10 min.

TABLE 23
Absorption of formulations suspended in water

Absorbance at 400 nm

| Formulation # | Initial | 10 min |
|---|---|---|
| 2 | 0.7705 | 0.6010 |
| 3 | 1.2312 | 1.1560 |
| 4 | 3.1265 | 3.1265 |

To further assess the formulations of calcitriol, a solubility study was conducted to evaluate the amount of calcitriol soluble in each formulation. Calcitriol concentrations from 0.1 to 0.6 μg calcitriol/mg formulation were prepared by heating the formulations to 50° C. followed by addition of the appropriate mass of calcitriol. The formulations were then allowed to cool to room temperature and the presence of undissolved calcitriol was determined by a light microscope with and without polarizing light. For each formulation, calcitriol was soluble at the highest concentration tested, 0.6 μg calcitriol/mg formulation.

A 45 μg calcitriol dose is currently being used in Phase 2 human clinical trials. To develop a capsule with this dosage each formulation was prepared with 0.2 μg calcitriol/mg formulation and 0.35% w/w of both BHA and BHT. The bulk formulation mixtures were filled into Size 3 hard gelatin capsules at a mass of 225 mg (45 μg calcitriol). The capsules were then analyzed for stability at 5° C., 25° C./60% relative humidity (RH), 30° C./65% RH, and 40° C./75% RH. At the appropriate time points, the stability samples were analyzed for content of intact calcitriol and dissolution of the capsules. The calcitriol content of the capsules was determined by dissolving three opened capsules in 5 mL of methanol and held at 5° C. prior to analysis. The dissolved samples were then analyzed by reversed phase HPLC. A Phemonex Hypersil BDS C18 column at 30° C. was used with a gradient of acetonitrile from 55% acetonitrile in water to 95% acetonitrile at a flow rate of 1.0 mL/min during elution. Peaks were detected at 265 nm and a 25 μL sample was injected for each run. The peak area of the sample was compared to a reference standard to calculate the calcitriol content as reported in Table 24. The dissolution test was performed by placing one capsule in each of six low volume dissolution containers with 50 mL of deionized water containing 0.5% sodium dodecyl sulfate. Samples were taken at 30, 60 and 90 min after mixing at 75 rpm and 37° C. Calcitriol content of the samples was determined by injection of 100 μL samples onto a Betasil C18 column operated at 1 mL/min with a mobile phase of 50:40:10 acetonitrile:water:tetrahydrofuran at 30° C. (peak detection at 265 nm). The mean value from the 90 min dissolution test results of the six capsules was reported (Table 25).

TABLE 24
Chemical stability of calcitriol formulation in hard gelatin capsules
(225 mg total mass filled per capsule, 45 μg calcitriol)

| Storage | Time | Assay^{a}(%) | | | |
|---|---|---|---|---|---|
| Condition | (mos) | Form. 1 | Form. 2 | Form 3 | Form 4 |
| N/A | 0 | 100.1 | 98.8 | 99.1 | 100.3 |
| 5° C. | 1.0 | 99.4 | 98.9 | 98.9 | 104.3 |
| 25° C./60% RH | 0.5 | 99.4 | 97.7 | 97.8 | 102.3 |
| | 1.0 | 97.1 | 95.8 | 97.8 | 100.3 |
| | 3.0 | 95.2 | 93.6 | 96.8 | 97.9 |
| 30° C./65% RH | 0.5 | 98.7 | 97.7 | 96.8 | 100.7 |
| | 1.0 | 95.8 | 96.3 | 97.3 | 100.4 |
| | 3.0 | 94.2 | 93.6 | 95.5 | 93.4 |
| 40° C./75% RH | 0.5 | 96.4 | 96.7 | 98.2 | 97.1 |
| | 1.0 | 96.1 | 98.6 | 98.5 | 99.3 |
| | 3.0 | 92.3 | 92.4 | 93.0 | 96.4 |

^{a}Assay results indicate % of calcitriol relative to expected value based upon 45 μg content per capsule. Values include pre-calcitriol which is an active isomer of calcitriol.

TABLE 25
Physical Stability of Calcitriol Formulation in Hard Gelatin Capsules
(225 mg total mass filled per capsule, 45 μg calcitriol)

| Storage | Time | Dissolution^{a} (%) | | | |
|---|---|---|---|---|---|
| Condition | (mos) | Form. 1 | Form. 2 | Form 3 | Form 4 |
| N/A | 0 | 70.5 | 93.9 | 92.1 | 100.1 |
| 5° C. | 1.0 | 71.0 | 92.3 | 96.0 | 100.4 |
| 25° C./60% RH | 0.5 | 65.0 | 89.0 | 90.1 | 98.3 |
| | 1.0 | 66.1 | 90.8 | 94.5 | 96.2 |
| | 3.0 | 64.3 | 85.5 | 90.0 | 91.4 |
| 30° C./65% RH | 0.5 | 62.1 | 88.8 | 91.5 | 97.9 |
| | 1.0 | 65.1 | 89.4 | 95.5 | 98.1 |
| | 3.0 | 57.7 | 86.4 | 89.5 | 88.8 |
| 40° C./75% RH | 0.5 | 91.9 | 90.2 | 92.9 | 93.1 |
| | 1.0 | 63.4 | 93.8 | 94.5 | 95.2 |
| | 3.0 | 59.3 | 83.6 | 87.4 | 91.1 |

^{a}Dissolution of capsules was performed as described and the % calcitriol is calculated based upon a standard and the expected content of 45 μg calcitriol per capsule. The active isomer, pre-calcitriol, is not included in the calculation of % calcitriol dissolved. Values reported are from the 90 min sample.

The chemical stability results indicated that decreasing the MIGLYOL 812 content with a concomitant increase in Vitamin E TPGS content provided enhanced recovery of intact calcitriol as noted in Table 24. Formulation 4 (50:50 MIGLYOL 812/Vitamin E TPGS) was the most chemically stable formulation with only minor decreases in recovery of intact calcitriol after 3 months at 25° C./60% RH, enabling room temperature storage.

The physical stability of the formulations was assessed by the dissolution behavior of the capsules after storage at each stability condition. As with the chemical stability, decreasing the MIGLYOL 812 content and increasing the Vitamin E TPGS content improved the dissolution properties of the formulation (Table 25). Formulation 4 (50:50 MIGLYOL 812Nitamin E TPGS) had the best dissolution properties with suitable stability for room temperature storage.

### EXAMPLE 17

### Pharmacokinetics of Formulation 4

Experiments were performed to compared the pharmacokinetic characteristics of Formula 4 (#4) from Example 16 with semi-solid #3 (SS3). Calcitriol was prepared in the #4 and SS3 formulations and formulated as capsules containing 4.5 μg of calcitriol per capsule. Single capsules were administered orally to 20 male beagle dogs (a dose of approximately 0.5 μg/kg body weight). Half of the dogs were given the #4 capsule on day 1 and the SS3 capsule on day 7. The other 10 dogs received the SS3 capsule on day 1 and the #4 capsule on day 7. Blood was collected 60, 40, and 20 minutes before each dose and 0.5, 1, 1.5, 2, 3, 4, 6, 8, 10, 12, 24, 36, 48, and 96 hours after each dose. Pharmacokinetic analysis of calcitriol levels in the blood samples was performed and the results shown in Table 26.

TABLE 26
Comparison of pharmacokinetics of calcitriol in the SS3 and #4
formulations.

| | Cₘₐₓ | AUC₍₀₋ₜ₎ | AUC_{(0-∞)} |
|---|---|---|---|
| Formulation | (pg/mL) | (pg · h/mL) | (pg · h/mL) |
| SS3 | 1125 | 10061 | 11341 |
| #4 | 1075 | 10269 | 11228 |

As can be seen from the data, the #4 and SS3 formulations exhibit very similar pharmacokinetics and thus are bioequivalent.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

1. A pharmaceutical composition comprising:
(a) a lipophilic phase component,
(b) one or more surfactants, and
(c) an active vitamin D compound;
wherein said composition comprises one of the following combinations of lipophilic phase component and one or more surfactants, wherein the percentage of each component is by weight based upon the total weight of the composition excluding the active vitamin D compound:

| | | |
|---|---|---|
| a. | Gelucire 44/14 | about 50% |
| | MIGLYOL 812 | about 50%; |
| b. | Gelucire 44/14 | about 50% |
| | Vitamin E TPGS | about 10% |
| | MIGLYOL 812 | about 40%; |
| c. | Gelucire 44/14 | about 50% |
| | Vitamin E TPGS | about 20% |
| | MIGLYOL 812 | about 30%; |
| d. | Gelucire 44/14 | about 40% |
| | Vitamin E TPGS | about 30% |
| | MIGLYOL 812 | about 30%; |
| e. | Gelucire 44/14 | about 40% |
| | Vitamin E TPGS | about 20% |
| | MIGLYOL 812 | about 40%; |
| f. | Gelucire 44/14 | about 30% |
| | Vitamin E TPGS | about 30% |
| | MIGLYOL 812 | about 40%; |
| g. | Gelucire 44/14 | about 20% |
| | Vitamin E TPGS | about 30% |
| | MIGLYOL 812 | about 50%; |
| h. | Vitamin E TPGS | about 50% |
| | MIGLYOL 812 | about 50%; |
| i. | Gelucire 44/14 | about 60% |
| | Vitamin E TPGS | about 25% |
| | MIGLYOL 812 | about 15%; |
| j. | Gelucire 50/13 | about 30% |
| | Vitamin E TPGS | about 5% |
| | MIGLYOL 812 | about 65%; |
| k. | Gelucire 50/13 | about 50% |
| | MIGLYOL 812 | about 50%; |
| l. | Gelucire 50/13 | about 50% |
| | Vitamin E TPGS | about 10% |
| | MIGLYOL 812 | about 40%; |
| m. | Gelucire 50/13 | about 50% |
| | Vitamin E TPGS | about 20% |
| | MIGLYOL 812 | about 30%; |
| n. | Gelucire 50/13 | about 40% |
| | Vitamin E TPGS | about 30% |
| | MIGLYOL 812 | about 30%; |
| o. | Gelucire 50/13 | about 40% |
| | Vitamin E TPGS | about 20% |
| | MIGLYOL 812 | about 40%; |
| p. | Gelucire 50/13 | about 30% |
| | Vitamin E TPGS | about 30% |
| | MIGLYOL 812 | about 40%; |
| q. | Gelucire 50/13 | about 20% |
| | Vitamin E TPGS | about 30% |
| | MIGLYOL 812 | about 50%; |
| r. | Gelucire 50/13 | about 60% |
| | Vitamin E TPGS | about 25% |
| | MIGLYOL 812 | about 15%; |
| s. | Gelucire 44/14 | about 50% |
| | PEG 4000 | about 50%; |
| t. | Gelucire 50/13 | about 50% |
| | PEG 4000 | about 50%; |
| u. | Vitamin E TPGS | about 50% |
| | PEG 4000 | about 40%; |
| v. | Gelucire 44/14 | about 33.3% |
| | Vitamin E TPGS | about 33.3% |
| | PEG 4000 | about 33.3%; |
| w. | Gelucire 50/13 | about 33.3% |
| | Vitamin E TPGS | about 33.3% |
| | PEG 4000 | about 33.3%; |
| x. | Gelucire 44/14 | about 50% |
| | Vitamin E TPGS | about 50%; |
| y. | Gelucire 50/13 | about 50% |
| | Vitamin E TPGS | about 50%; |
| z. | Vitamin E TPGS | about 5% |
| | MIGLYOL 812 | about 95%; |
| aa. | Vitamin E TPGS | about 5% |
| | MIGLYOL 812 | about 65% |
| | PEG 4000 | about 30%; |
| ab. | Vitamin E TPGS | about 10% |
| | MIGLYOL 812 | about 90%; |
| ac. | Vitamin E TPGS | about 5% |
| | MIGLYOL 812 | about 85% |
| | PEG 4000 | about 10%; and |
| ad. | Vitamin E TPGS | about 10% |
| | MIGLYOL 812 | about 80% |
| | PEG 4000 | about 10%. |

2. A pharmaceutical composition comprising an active vitamin D compound, about 50% MIGLYOL 812, about 50% vitamin E TPGS, about 0.35% butylated hydroxy anisole (BHA), and about 0.35% butylated hydroxytoluene (BHT).

3. The pharmaceutical composition of claims 1 or 2, wherein said active vitamin D compound is calcitriol.

4. The pharmaceutical composition of claim 1, further comprising at least one additive selected from the group consisting of an antioxidant, a bufferant, an antifoaming agent, a detackifier, a preservative, a chelating agent, a viscomodulator, a tonicifier, a flavorant, a colorant, an odorant, an opacifier, a suspending agent, a binder, a filler, a plasticizer, a thickening agent, and a lubricant.

5. The pharmaceutical composition of claim 4, wherein one of said additives is an antioxidant.

6. The pharmaceutical composition of claim 5, wherein said antioxidant is selected from the group consisting of ascorbic acid, ascorbyl palmitate, BHA, BHT, potassium metabisulfite, sodium bisulfite, sodium metabisulfite, and tocopherol.

7. The pharmaceutical composition of claims 1 or 2 adapted for oral administration.

8. The pharmaceutical composition of claim 7 in unit dosage form.

9. The pharmaceutical composition of claim 8 comprising 1-400 μg of an active vitamin D compound per said unit dose.

10. The pharmaceutical composition of claim 9 comprising 45 μg of an active vitamin D compound per said unit dose.

11. The pharmaceutical composition of claim 9, wherein said active vitamin D compound is calcitriol.

12. The pharmaceutical composition of claim 8, wherein said unit dosage form is a capsule.

13. The pharmaceutical composition of claim 12, wherein said capsule is a gelatin capsule.

14. The pharmaceutical composition of claim 13, wherein the total volume of ingredients present in said gelatin capsule is 10-1000 μL.

15. The pharmaceutical composition of claim 13, wherein the total weight of ingredients present in said gelatin capsule is 10-1500 mg.

16. A method for the treatment or prevention of a hyperproliferative disease, said method comprising administering the pharmaceutical composition of claims 1 or 2 to a patient in need thereof.

17. The method of claim 16, wherein said hyperproliferative disease is cancer.

18. The method of claim 16, wherein said hyperproliferative disease is psoriasis.

19. The method of claim 16, wherein the pharmaceutical composition is administered by pulse-dose, wherein said pulse-dose comprises the administration of said composition to a patient no more than once every three days.

20. The method of claim 19, wherein said administration is no more than once a week.

21. The method of claim 20, wherein said administration is no more than once every three weeks.

22. The method of claim 16, further comprising administering one or more chemotherapeutic agents or radiotherapeutic agents/treatments.

23. The method of claim 22, wherein said active vitamin D compound is administered at least 12 hours prior to the administration of said one or more chemotherapeutic agents or radiotherapeutic agents/treatments.

24. The method of claim 23, wherein said active vitamin D compound is administered for 1 day to about 3 months prior to the administration of said one or more chemotherapeutic agents or radiotherapeutic agents/treatments.

25. The method of claim 22, wherein said active vitamin D compound is administered concurrently with the administration of said one or more chemotherapeutic agents or radiotherapeutic agents/treatments.

26. The method of claim 25, wherein the administration of said active vitamin D compound is continued beyond the administration of said one or more chemotherapeutic agents or radiotherapeutic agents/treatments.

27. The method of claim 22, wherein the active vitamin D compound is administered after the administration of said one or more chemotherapeutic agents or radiotherapeutic agents/treatments.

28. The method of claim 22, wherein said active vitamin D compound is administered 1 day prior to the administration of said one or more chemotherapeutic agents or radiotherapeutic agents/treatments.

29. The method of claim 22, wherein said active vitamin D compound and said one or more chemotherapeutic agents or radiotherapeutic agents/treatments are administered no more than once every three weeks.
